(19) United States
(12) Patent Application Publication
(10) Pub. No.: US 2023/0398061 A1
(43) Pub. Date: Dec. 14, 2023

(54) INJECTABLE HYDROGELS FOR ADOPTIVE CELL THERAPY

(71) Applicant: The Board of Trustees of the Leland Stanford Junior University, Stanford, CA (US)

(72) Inventors: Abigail Kate GROSSKOPF, Stanford, CA (US); Eric Andrew APPEL, Stanford, CA (US); Crystal MACKALL, Stanford, CA (US); Louai LA-BANIEH, Stanford, CA (US)

(73) Assignee: The Board of Trustees of the Leland Stanford Junior University, Stanford, CA (US)

(21) Appl. No.: 18/249,708

(22) PCT Filed: Oct. 20, 2021
(86) PCT No.: PCT/US2021/055897

§ 371 Date: Apr. 19, 2023

Related U.S. Application Data
(60) Provisional application No. 63/094,243, filed on Oct. 20, 2020.

(51) Int. Cl. A61K 9/00; A61K 47/66; A61K 39/00; A61K 38/19; A61K 47/36; A61K 47/34

(52) U.S. Cl. CPC A61K 9/0024; A61K 47/66; A61K 39/4611; A61K 39/4631; A61K 38/19; A61K 47/36; A61K 47/34; A61K 39/4644; A61K 39/464411; A61K 39/4622; A61K 39/4613

(57) ABSTRACT

An immunotherapy delivery system includes a hydrogel with an immunomodulatory cargo including cells encapsulated in the hydrogel, a cell adhesion motif in the hydrogel configured to reversibly adhere to and release the cells, and an immunomodulatory cargo encapsulated in the hydrogel. The hydrogel includes a polymer non-covalently crossed-linked with a plurality of nanoparticles.

### CROSS REFERENCE TO RELATED APPLICATIONS

This application claims priority to U.S. Provisional Patent Application No. 63/094,243 filed Oct. 20, 2020 and entitled “Injectable Hydrogels for Adoptive Cell Therapy,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are systems, methods, and compositions (including kits) utilizing a hydrogel for delivering cells and immunomodulatory cargo. These methods and compositions may be particularly useful as an immunotherapy niche for treating cancer and especially for treating solid tumors.

### BACKGROUND

An estimated 18.2 million Americans, or roughly 1 in 19 people, are cancer patients or cancer survivors. Many of these cancers are solid tumors. Cellular engineering approaches have been developed to target therapeutic cells to solid tumors in cancer patients. In one approach, therapeutic cells are delivered into the blood through an infusion process. However, solid tumors have dense harsh environments with many checkpoint inhibitors and infused therapeutic cells may not find and infiltrate these solid tumors.

Therapeutic cells for cancer treatment can include immune cells from the patient. Adoptive cell therapy (ACT) is a promising strategy to treat cancer. In the ACT process, immune cells are collected from a patient, isolated, and engineered with receptors to help combat cancer, expanded, and then infused into the patient as the treatment. Chimeric antigen receptor (CAR) T cells are immune cells engineered to target an antigen that is over-expressed on cancer cells. Use of ACT has been widely effective in treating blood cancers, including B-cell leukemias and lymphomas, but has previously seen limited success in treating solid tumors. Several therapies for treating B-cell malignancies have recently been approved by the United States Food & Drug Administration (U.S. FDA), and numerous concerted efforts have been underway to translate this success to solid tumor applications. Unfortunately, there has been limited success, because, for example, large numbers of CAR-T cells are typically required for these treatment strategies to be successful, which requires costly, complex, and labor-intensive ex vivo expansion of cells for a relatively long time, such as e.g., 7-10 days. Moreover, extended ex vivo expansion of cells can often limit the effector potential of transferred CAR-T cells. Thus, strategies for reducing the ex vivo expansion time by decreasing the required CAR-T cell dose would help in the clinical translation and broad dissemination of CAR-T therapy. Additionally, reducing the dose of cells needed for treatment would likely reduce the severe side effects previously associated with treatment, including, for example, cytokine storm and neurotoxicity.

Currently, CAR-T cells are delivered through intravenous (IV) infusion through blood. This can be effective in treating blood cancers but not solid tumors because the T cells do not need to find and penetrate a solid tumor microenviroment to treat blood cancers. Unfortunately, T cells administered through intravenous infusion also often become trapped in the lungs and exhibit poor infiltration of solid tumors. Use of locoregional cell delivery methods (particularly those exploiting biomaterial scaffolds) for delivery of T cells near a tumor, have shown improved local expansion of T cells at the tumor site, improving tumor infiltration and enhancing treatment of solid tumors. Unfortunately, the biomaterials scaffolds developed for locoregional ACT have limitations, such as requiring invasive surgical implantation procedures to reach tumor sites, which hinders translation and use of these methods.

Additionally, CAR-T cells have to be activated, and CAR-T cells may be most effective when fully activated state at the tumor site. The high local cytokine concentrations required for particular CAR-T activation are highly toxic if delivered systemically. Currently, T cells are expanded in high concentrations of cytokines only prior to delivery to the patient to avoid these toxicities. Locoregional approaches for delivering cytokines would limit systemic cytokine exposure and reduce toxicities, and as such exhibit the potential to maintain adoptive T cells in a highly activated state in vivo.

Furthermore, T cell exhaustion describes the progressive loss of effector function in T cells due to prolonged antigen stimulation (e.g., by cytokines) and T cell exhaustion can lead to patient relapse. Efficient T cell activation generally requires three signals: T cell receptor signaling (1), activation by co-stimulatory molecules (2), and immune stimulatory cytokines (3). Signals (1) and (2) have been achieved through some cellular engineering approaches, but signal (3) is still largely unmet with current treatment strategies. T cell persistence can be an important clinical determinant for durable response and has been especially poor in clinical trials of solid tumors. Thus, multiple strategies have been explored for supplementing T cells with cytokines or cytokine signaling domains to increase T cell persistence. In their native state, cytokines are highly local signaling events, and, if delivered in high concentration through blood infusion to a patient, cytokines can be highly toxic. IL-15, IL-2, IL-12, and IL-7 cytokines have all shown promising results but distinct mechanisms towards aiding CAR-T therapies. Local approaches to delivering cytokines have shown some efficacy, but the techniques have required extensive cytokine-engineering and the presence of certain biological signatures that bind to the cytokines. Biomaterials and hydrogels present an exciting engineering opportunity to locally expose cells to stimulatory factors to create artificial environments similar to that of the lymph node which normally modulates the cells.

Accordingly, there is a need for new treatments to address these and other limitations. Described herein are methods and devices that may address these and other limitations.

### SUMMARY OF THE DISCLOSURE

Described herein are systems, methods, and compositions (including kits) utilizing a hydrogel useful for encapsulating and delivering an immunomodulatory cargo such as cells and other cargo. The cells and other cargo may be immunomodulatory components delivered to a patient in need of immunotherapy. The hydrogel system can include a cell adhesion motif configured for reversibly adhering and releasing the cells and allowing the cells to move through and exit the hydrogel. When delivered to a patient, these systems may act as cell niches (e.g., immune cell niches for immune cells), slowly releasing and delivering cells to the patient over a period of time (e.g., hours, days, weeks, months). The hydrogel system may also contain a second immunomodulatory cargo (e.g., cytokines) for stimulating the cells in vivo and may do so prior to cell release from the hydrogel. The hydrogel may be physically crosslinked and have relatively small pores/mesh. The hydrogel system may surprisingly be configured to keep some cargo such as cytokines in the hydrogel while facilitating and controlling the release of molecules much larger than the cytokines, such as the cells. For some immunomodulatory cargo, such as cytokines, the relatively small pores/mesh size of the hydrogel can keep the immunomodulatory cargo from diffusing out of the hydrogel (e.g., even when the immunomodulatory cargo is not attached to the hydrogel). As some molecules, such as certain concentrations of cytokines, can be toxic to patients and cause a wide range of adverse effects, the hydrogel system described herein may facilitate delivery of therapeutically useful amounts of immune cells without associated toxicity. The systems may allow diffusion of smaller nutrients to maintain the cells. These methods and compositions may be particularly useful as an immunotherapy niche for treating cancer and especially for treating solid tumors.

In addition to reaching the tumor in high numbers, the cells from the hydrogel system may also need to be activated and timing of activation and activation signals can be important. T cell exhaustion describes the progressive loss of effector function in T cells due to prolonged antigen stimulation (e.g., by cytokines) and T cell exhaustion can lead to relapse. Efficient T cell activation generally requires three signals: T cell receptor signaling (1), activation by co-stimulatory molecules (2), and immune stimulatory cytokines (3). Signals (1) and (2) have been achieved through cellular engineering approaches, but signal 3 is still largely unmet with current treatment strategies. T cell persistence can be a critical clinical determinant for durable response and has been especially poor in clinical trials of solid tumors. Thus, multiple strategies are being explored for supplementing T cells with cytokines or cytokine signaling domains to increase their persistence. In their native state, cytokines are highly local signaling events, and, if delivered in high concentration through infusion to a patient, cytokines can be highly toxic. IL-15, IL-2, IL-12, and IL-7 have all showed promising results but distinct mechanisms towards aiding CAR-T therapies. Local approaches to delivering of cytokines have shown efficacy, but techniques have required extensive cytokine-engineering and the presence of certain biological signatures that bind to the cytokines. Biomaterials and hydrogels present an exciting engineering opportunity to locally expose cells to stimulatory factors to create artificial environments similar to that of the lymph-node. Previous biomaterials approaches have conjugated activating signals to the materials for both in vitro expansion and in vivo treatment, but, we propose that, if the hydrogel mesh size is engineered to be small enough, it is possible to simply mix stimulatory molecules in the material while still achieving local signaling and slowed diffusion.

Described herein is a new method for CAR-T cell delivery based on injectable Polymer-Nanoparticle (PNP) hydrogels. These hydrogels may advantageously utilize scalable chemistry and rapid formulation to encapsulate and deliver cells (locally) to tumor sites through simple injection (see FIG. 1E). With a smaller pore size compared to most hydrogel systems that limits diffusion, these hydrogels can retain local signals to activate adoptive cells through simple mixing. With this modular system, various cytokines can be incorporated into treatment enabling more modular personalized treatment. Supramolecular transient interactions that hold the hydrogel network together can allow for fast (instantaneous) self-healing after injection and access to reach tumors in many parts of the body through injection or catheter delivery. Increased efficacy in fighting tumors was demonstrated compared to traditional methods with the novel cell delivery strategies described herein.

In general, an immunotherapy delivery system includes a hydrogel, a first immunomodulatory cargo including cells encapsulated in the hydrogel, a cell adhesion motif in the hydrogel configured to reversibly adhere to and release the cells, and a second immunomodulatory cargo encapsulated in the hydrogel. The hydrogel includes a polymer non-covalently crossed-linked with a plurality of nanoparticles.

This and other embodiments can include one or more of the following features. The cell adhesion motif can include a peptide configured to reversibly adhere to and release the cells. The cell adhesion motif can be configured to bind to integrins on the cells. The cell adhesion motif can include an arginine-glycine-aspartic acid (RGD) peptide. The nanoparticles can include the cell adhesion motif. The nanoparticles can be configured to present the cell adhesion motif.

This and other embodiments can include one or more of the following features. The cells can include adoptive cells. The cells can include chimeric antigen receptor (CAR) T cells or chimeric antigen receptor (CAR) natural killer cells.

This and other embodiments can include one or more of the following features. The second immunomodulatory cargo can include a protein. The second immunomodulatory cargo can include a cytokine.

This and other embodiments can include one or more of the following features. The hydrogel can include less than 5% polymer. The hydrogel can include 1.5%-3% polymer. The hydrogel can include approximately 2% polymer. The polymer can include hydroxypropylmethylcellulose (HPMC). The polymer can include hydroxypropylmethylcellulose (HPMC) with hydrophobic lipid dodecyl chains.

The nanoparticles can include poly(ethylene glycol)-bpoly(lactic acid) (PEG-PLA). The nanoparticles can include the cell adhesion motif attached to the poly(ethylene glycol)-bpoly(lactic acid) (PEG-PLA). The nanoparticles can include between a 10:90 and a 90:10 ratio of poly(ethylene glycol)-bpoly(lactic acid) (PEG-PLA) with a cell adhesion motif to poly(ethylene glycol)-bpoly(lactic acid) (PEG-PLA) without a cell adhesion motif. The nanoparticles can include between a 25:75 ratio and a 75:25 of poly(ethylene glycol)-bpoly(lactic acid) (PEG-PLA) with a cell adhesion motif to poly(ethylene glycol)-bpoly(lactic acid) (PEG-PLA) without with a cell adhesion motif.

The hydrogel can include 4-12% nanoparticles. The hydrogel can be shear-thinning and self-healing.

This and other embodiments can include one or more of the following features. The immunotherapy delivery system can include a syringe or catheter containing the hydrogel.

In general, a method of treating a disease, includes delivering any of the immunotherapy delivery systems described above to a patient; and releasing the cells from the hydrogel into the patient.

This and other methods can include the step of releasing the cells from the hydrogel over a period lasting from one day to four weeks.

This and other methods can include wherein the immunotherapy delivery system releases cells over the course of at least two weeks, at least three weeks, or at least four weeks.

This and other methods can further include the step of activating the cells with second immunomodulatory cargo. This and other methods can further include the step of expanding the number of cells in the hydrogel.

In this and other methods, the disease can be a solid tumor cancer.

In this and other methods, the cells can be autologous. In this and other methods, the cells can be autogeneic.

In this and other methods, the cells can express a chimeric antigen receptor (CAR) that recognizes a tumor antigen.

This and other methods can further include one or more of the steps of removing the cells from the patient or a donor; isolating the removed cells; modifying the removed cells (e.g., with a receptor); expanding the number of cells in vitro; and/or encapsulating the cells in the hydrogel prior to the delivering step.

In this and other methods, the cells can successively attach to and detach from the cell adhesion motif in the hydrogel.

In this and other methods, delivering can include delivering the immunotherapy delivery system through a syringe or catheter.

In this and other methods, delivering can include delivering the immunotherapy delivery system to the patient by a route selected from the group consisting of intravenous, intraperitoneal, intramuscular, intratumoral, and subcutaneous.

In this and other methods, delivering can include delivering the immunotherapy delivery system to the patient locally to a region of the patient needing treatment. In this and other methods, delivering can include delivering the immunotherapy delivery system to a solid tumor cancer in the patient.

In this and other methods, delivering can include delivering the immunotherapy delivery system to the patient. In this and other methods, delivering can include delivering the system to a location remote from a region of the patient needing treatment. In this and other methods, wherein the disease includes a solid tumor cancer, delivering the immunotherapy delivery system to the patient can include delivering the system to a location in the patient remote from the solid tumor cancer. In this and other methods, delivering the immunotherapy delivery system to the patient can include delivering the system systemically to the patient.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1T illustrate the formation of Polymer-Nanoparticle (PNP) hydrogels. The PNP hydrogels can control CAR-T cell migration and release of small cytokines.

FIGS. 1A-1C illustrate formation of PNP hydrogels to encapsulate CAR-T cells and stimulatory cytokines through self-assembly of biopolymers and degradable nanoparticles.

FIG. 1A schematically illustrates formation of a dodecyl modified hydroxypropylmethylcellulose (HPMC-C12) starting material.

FIG. 1B schematically illustrates formation of poly poly(ethylene glycol)-block polylactide (PEG-b-PLA) starting material.

FIG. 1C schematically illustrates a process for encapsulating cells and modulators in an injectable hydrogel for use in immunotherapy.

FIG. 1D schematically illustrates a traditional intravenous (IV) infusion showing systemic administration of cells for cancer treatment.

FIG. 1E schematically illustrates an example of local administration of cells using an immunotherapy delivery system as described herein with cells and a modulator encapsulated in a hydrogel for controlled delivery to a patient.

FIGS. 1F-1R schematic illustrate a delivery method for delivering CAR-T cells to solid tumors.

FIG. 1F shows a schematic illustration of the B7H3 CAR construct used for all studies.

FIG. 1G shows B7H3 CAR-T cells are efficiently transduced. Transduction efficiency of B7H3 CAR-T Cells is determined by staining with B7H3-Fc.

FIG. 1H shows formulation of PNP hydrogels by mixing of the biopolymer solution in one syringe (left) with a solution of RGD-modified nanoparticles, cells, and cytokines in the other syringe (right) using a luer lock mixer.

FIG. 1I shows that after gentle mixing of the solutions as shown in FIG. 1H for 30 seconds, a solid-like PNP hydrogel encapsulating cells homogeneously is formed (right syringe).

FIG. 1J shows injection of cell-loaded PNP hydrogel through a 26 G needle onto a substrate.

FIG. 1K shows a robust, solid-like hydrogel depot is formed on the substrate shown in FIG. 1J and the depot does not significantly flow due to gravity.

FIG. 1L, shows a schematic illustration of an in vitro experimental set-up to evaluate CAR-T cell motility within PNP hydrogels within indicated formulations.

FIGS. 1M-1O illustrate trajectories of migrating CAR-T cells in different hydrogel compositions. FIG. 1M illustrates trajectories of migrating CAR-T cells in a PNP-1-1 hydrogel. FIG. 1N illustrates trajectories of migrating CAR-T cells in a PNP-1-5 hydrogel. FIG. 1O illustrates trajectories of migrating CAR-T cells in a PNP-2-10 hydrogel. PNP-1-1, PNP-1-5, PNP-2-10 hydrogel formulations were tested, where the first number is the wt % HPMC-C₁₂ polymer and the second number is the wt % NPs. The trajectories were plotted at a common origin for easy visualization where each grid is 50 μm wide. FIG. 1P shows CAR-T cell migration speeds in different PNP hydrogel formulations as quantified through cell migration experiments (n>150 cells for all samples; mean±s.e.m.).

FIGS. 1Q-1T illustrate that the PNP system described herein can release a controlled amount of CAR-T cells over time. FIG. 1Q schematically illustrates a testing device for determining cell release over time. 1 million CAR-T cells were loaded into three different hydrogel formulations and a media bolus group. The number of cells released over time from each sample were counted. FIG. 1R shows the cumulative number of cells released over time from the setup shown in FIG. 1Q. FIG. 1S shows that PNP 1-5 contains the greatest number of cells after 8 days. FIG. 1S shows the number of cells remaining at Day eight in each of the three different hydrogel formulations tested in the testing device shown in FIG. 1Q. FIG. iT shows cells in the three different hydrogel formulations tested in the testing device shown in FIG. 1Q remain viable.

FIG. 2A shows improved survival and tumor reduction or elimination after treatment of animals with CAR-T cells using the hydrogel delivery system described herein. FIG. 2A shows results of whole animal luminescent imaging for tumors over time in an animal model with luminescent medulloblastoma tumor after immunotherapy treatment with chimeric antigen receptor (CAR) T cells. After treatment with chimeric antigen receptor (CAR) T cells in hydrogel having a polymer non-covalently cross-linked with nanoparticles or with PBS, tumors were undetectable. Untreated animals died.

FIG. 2B shows robust chimeric antigen receptor (CAR) T cell proliferation from CAR-T cells delivered using the polymer-nanoparticle hydrogel delivery systems described herein. FIG. 2B also shows migration of the CAR-T cells to target tumors. Whole animals were imaged to detect luminescent chimeric antigen receptor (CAR) T cells over time after the cells were delivered to an animal medulloblastoma tumor model.

FIG. 2C shows treating animals with chimeric antigen receptor (CAR) T cells using the polymer-nanoparticle hydrogel delivery systems described herein eliminates tumors or reduces them to undetectable. FIG. 2C shows tumor luminescence quantification over time with various treatments. Each line represents an individual mouse. The shading at the bottom part of the graph represents the average background noise during sampling.

FIG. 3A shows that including IL-15 cytokine in the hydrogel leads to tumor reduction or elimination after treatment of animals with chimeric antigen receptor (CAR) T cells using the polymer-nanoparticle hydrogel delivery system described herein. 2*10⁶ chimeric antigen receptor (CAR) T cells were delivered to medulloblastoma tumor in an animal model with various treatment strategies and the animals subject to luminescence imaging.

FIG. 3B shows quantification of the tumor luminescence over time for various treatments. Each line represents an individual mouse. The shading at the bottom part of the graph represents the average background noise during sampling.

FIG. 4A-FIG. 4B shows that treating animals from an animal medulloblastoma tumor model with chimeric antigen receptor (CAR) T cells using the polymer-nanoparticle hydrogel delivery systems described herein delivered remote from the tumor eliminates tumors or reduces them to undetectable. FIG. 4A shows whole animal imaging over time to detect tumors after systemic delivery of the chimeric antigen receptor (CAR) T cells with the polymer-nanoparticle hydrogel delivery. FIG. 4B shows quantification of the tumor luminescence over time after systemic delivery of the chimeric antigen receptor (CAR) T cells with the polymer-nanoparticle hydrogel delivery. Each line represents an individual mouse.

FIG. 5A-FIG. 5K shows that PNP hydrogels improve treatment efficacy and CAR-T cell expansion. FIG. 5A shows an experimental timeline for cancer experiments fir treating subcutaneous human medulloblastoma in mice with 2×106 CAR-T cells administered with different delivery methods, including: (i) i.v. bolus, (ii) s.c. bolus, (iii) s.c. bolus delivery containing 0.25 ug IL-15, (iv) PNP-1-5 hydrogel, and (v) PNP-1-5 hydrogel containing 0.25 μg IL-15. Results are shown in FIG. 5B-FIG. 5J.

FIG. 5B shows results of luminescent imaging of tumors in all experimental groups at all time points. FIG. 5C shows quantification of imaging data for all experimental groups, whereby red shading represents background signal of the data (n=8-10 for all groups over 2 experiments). FIG. 5D shows the number of days to cure for each treatment, defined as the time whereby the luminescent signal dropped and stayed below the background signal of the in vivo imaging apparatus (n=8-10 for all groups over 2 experiments; mean±s.d.). FIG. 5E shows results of luminescent imaging of CAR-T cells in all corresponding experimental groups. FIG. 5F shows quantification of imaging data for all experimental groups, whereby the shading at the bottom of the graph represents background signal of the data (n=3-6 for all groups over 2 experiments). FIG. 5G shows CAR-T cell luminescent signal after 21 days (n=3-6 for all groups over 2 experiments; mean±s.d.). FIG. 5H shows results for CAR MFI (n=3 for all groups; mean±s.d.). FIG. 5I shows total CD3+ T cells (n=3 replicates for all groups; mean±s.d.). FIG. 5J shows results of relative CD4+ and CD8+ content analysis (mean of n=3 for all groups). FIG. 5K shows results from memory CAR-T cell subsets (mean of n=3 replicates for all groups) of ex vivo expanded CAR-T cells extracted from PNP-1-5 gels 10 days after treatment in the MED8A tumor model.

FIG. 6A-FIG. 6F show PNP Hydrogels are effective in treating distal subcutaneous human medulloblastoma in mice.

FIG. 6A shows a schematic showing placement of subcutaneous tumor and distal subcutaneous treatments in mice. FIG. 6B shows a schematic illustration of experimental timeline and treatment placement whereby mice received either (i) distal s.c. bolus injection of 2×106 CAR-T cells and 0.25 μg IL-15, or (ii) PNP-1-5 hydrogel containing 2×106 CAR-T cells and 0.25 μg IL-15.

FIG. 6C-FIG. 6F shows results from the experiments illustrated in FIGS. 6A-6B. FIG. 6C shows results of representative luminescent imaging of tumors with in vivo imaging (n=5 mice for each group).

FIG. 6D and FIG. 6E shows results from quantification of imaging data for both experimental groups (distal bolus in FIG. 6D and PNP-1-5 hydrogel containing 2×106 CAR-T cells and 0.25 μg IL-15 in FIG. 6E), whereby shading at the bottom of the graph represents background signal of the data (n=5 mice).

FIG. 6F shows the number of days to cure for each treatment, defined as the time whereby the luminescent signal dropped below the background signal of the in vivo imaging apparatus; mean±s.d.).

FIG. 7 shows successful coupling of the dodecyl side chain to the HPMC. Trace A shows 1H-NMR (DMSO-d6) of dodecyl/-isothiocyanate starting material. Trace B shows 1H-NMR (DMSO-d6) of hypromellose (HPMC) starting material. Trace C shows 1H-NMR (DMSO-d6) of dodecyl-modified HPMC (HPMC-C12), showing the emergence of the terminal methyl group on the dodecyl side chain at 0.86 ppm, indicating the successful coupling of the dodecyl side chain to the HPMC.

FIG. 8A-FIG. 8D show rheology of PNP hydrogel formulations with varying polymer and nanoparticle weight percent. The first number represents the wt % polymer, and the second number represents the wt % nanoparticles. FIG. 8A shows frequency sweep (strain=1%) for all formulations at 25° C. FIG. 8B shows flow sweep for all formulations at 25° C. with steady state sensing up to 120 seconds. FIG. 8C shows amplitude sweep (w=10 rad/s) for all formulations at 25° C. FIG. 8D shows a summary of rheological parameters for the three formulations.

FIG. 9A-FIG. 9C shows rheology of the PNP-1-5 hydrogel formulations exploring the effect of using RGD-conjugated nanoparticles and encapsulating 20 million cells/mL. FIG. 9A shows frequency sweep (strain=1%) for all formulations at 25° C. FIG. 9B shows flow sweep for all formulations at 25° C. Amplitude sweep (w=10 rad/s) for all formulations at 25° C. FIG. 9C shows modulus as a function of strain.

FIG. 10 shows hydrogel matrix self-diffusivity for PNP-1-1, PNP-1-5, and PNP-2-10 hydrogel formulations comprising RGD-functional nanoparticles determined using fluorescence recovery after photobleaching (FRAP) experiments.

FIG. 11A-FIG. 11B show that PNP hydrogels improve stability of IL-15 under in vivo conditions. FIG. 11A shows results from a study examining the proportion of 0.25 μg IL-15 retained in 100 μL of PNP-1-5 hydrogel immersed in buffer over the course of 4 days. Data is normalized to the total amount collected over 4 days and the amount of IL-15 still remaining in the gel at 4 days. In this plot, the round points represent experimental data and the smooth line represents a one-phase exponential decay fit to the collected data. FIG. 11B shows that PNP hydrogels improve stability of IL-15 under in vivo conditions. In these studies, 0.25 μg of IL-15 was added to each sample, but only a percentage of that protein was found to remain active in different formulations due to the incubation of the samples at 37° C. The % active (compared to the amount added initially) IL-15 aging in saline for 4 days at 37° C., % active IL-15 collected over the course of the complete release study (over 4 days) at 37° C., and % active IL-15 that was collected on Day 4 during the release study from the gel (at 37° C.) showed that a higher proportion of the IL-15 collected during the release study remained active in the PNP hydrogel group. These studies demonstrate the powerful stabilizing effects of PNP hydrogels for IL-15.

FIG. 12 shows results of a WST Assay for assess the effect of IL-15 on CAR-T cell proliferation when encapsulated in PNP-1-5 hydrogels. Relative signal (absorbance was read using a plate reader at OD=450 nm) was reported to a control containing no IL-15. Data shown as mean±s.d. Based on this study, an IL-15 concentration of 2.5 μg/mL was used for in vivo studies described herein.

FIG. 13A-FIG. 13C shows RGD-conjugation to nanoparticles within the PNP hydrogel structure increases migration of CAR-T cells encapsulated in these hydrogels. FIG. 13A shows CAR-T cell speeds within PNP-1-5 hydrogel formulations with and without conjugation of RGD moieties (data shown as mean/pmSEM). FIG. 13B shows trajectories of migrating CAR-T cells within indicated hydrogel formulations of PNP-1-5 with RGD. FIG. 13C shows trajectories of migrating CAR-T cells within indicated hydrogel formulations of PNP-1-5 without RGD. The trajectories are plotted at a common origin for easy visualization. Each grid is 50 μm.

FIG. 14A-FIG. 14B show increased expression of PGC-1α, a master regulator of mitochondrial biogenesis, in IL-15 loaded PNP gels encapsulated at 20 million cells/mL and cultured for 3 days. MFI of PGC-1α staining in CD8+ T cells (FIG. 14A) and CD4+ T cells (FIG. 14B) encapsulated in various PNP hydrogel formulations. Data shown is mean±SEM. FIG. 15 shows results from a control group containing T cells with no CAR (Mock T cells) compared to CAR-T cells delivered in PNP-1-5 hydrogels with IL-15 at 20 million cells/mL. Tumor imaging using an in vivo imaging system. In these studies, n=5 from one experiment for the control group, while n=8 from two experiments (n=3 and n=5) for the PNP-1-5 IL-15 group.

FIG. 16A-FIG. 16D shows results of an In vivo experiment delivering 8 million CAR-T cells in PNP hydrogels and translationally-relevant controls. FIG. 16A shows results of tumor imaging using an in vivo imaging system (n=5 from one experiment for all groups). FIG. 16B shows results from corresponding quantification of luminescent signal from tumor imaging. FIG. 16C shows CAR-T cell imaging using an in vivo imaging system. FIG. 16D shows results from corresponding quantification of luminescent signal from CAR-T cell imaging (n=5 from one experiment for all groups). FIG. 17A-FIG. 17B shows pharmacokinetics of IL-15 in vivo with different delivery methods. FIG. 17A shows results with serum concentration of IL-15 over 48 hours delivered from a subcutaneous injection of PNP-1-5 hydrogel, a subcutaneous saline bolus, and an intravenous saline bolus. In these studies, a dose of 0.25 μg IL-15 was administered in all groups. Data shown as mean±s.d. (n=3 from one experiment for all groups).

FIG. 17B shows the corresponding Area-Under-the-Curve (AUC) for different delivery methods over the 48 hour evaluation period. Data shown as mean±s.d. (n=3 from one experiment for all groups).

FIG. 18 shows PNP1-5 IL-15 treatment increase cure. FIG. 18 shows the proportion of mice cured over the course of the experiment (n=8-10 mice). For these studies, “cured” was defined as exhibiting a luminescent tumor signal dropping below, and staying below, the background signal of the in vivo imaging apparatus.

FIG. 19A-FIG. 19F shows a significantly increased expansion of the cells resulting from PNP-1-5 IL-15 treatment. CAR-T cells (2 million) were co-delivered with IL-15 either intravenously or co-encapsulated within PNP hydrogels at a 0.25 μg dose of IL-15. FIG. 19A shows results from tumor imaging using an in vivo imaging system. FIG. 19B shows corresponding quantification of luminescent signal from tumor imaging (n=8 from two experiments for the PNP-1-5 IL-15 group; n=9 from one experiment for IV group). FIG. 19C shows the percent of mice that died on account of acute toxicity from each treatment. FIG. 19D shows predicted day to cure based on experimental data for each group. FIG. 19E shows CAR-T cell imaging using an in vivo imaging system (n=5 for all groups). FIG. 19F shows corresponding quantification of luminescent signal from CAR-T cell imaging (n=6 from two experiments for the PNP IL-15 group; n=5 for IV IL-15 group from one experiment). FIG. 19G shows the slopes of the CAR-T cell expansion within each experimental group demonstrating a significantly increased expansion of the cells resulting from the PNP-1-5 IL-15 treatment.

FIG. 20A-FIG. 20B shows results of an in vivo experiment comparing the co-delivery of 2 million CAR-T cells with IL-15 at a dose of either 0.25 μg/mouse or 2.5 μg/mouse in PNP hydrogels. FIG. 20A shows results from tumor imaging using an in vivo imaging system. FIG. 20B shows corresponding quantification of luminescent signal from tumor imaging. In these experiments, n=8 (n=5 and n=3) from two experiments for the 0.25 μg group, and n=5 from one experiment for the 2.5 μg group.

FIG. 21A-FIG. 21B shows results of in vivo experiment comparing the co-delivery of either 0.25 μg IL-2 or 0.25 μg IL-15 with CAR-T Cells (2 million) in PNP-1-5 hydrogel. FIG. 21A shows tumor imaging results using an in vivo imaging system. FIG. 21B shows corresponding quantification of luminescent signal from tumor imaging (n=8 from two experiments for the IL-15 group; n=5 from one experiment for IL-2 group). FIG. 21C shows results from CAR-T cell imaging using an in vivo imaging system (n=5 for all groups). FIG. 21D shows corresponding quantification of luminescent signal from CAR-T cell imaging. In these studies, n=6 (n=3 and n=3) from two experiments for the IL-15 group and n=5 from one experiment for IL-2 group. FIG. 22A-22F shows histology of explanted PNP-1-5 hydrogel containing CAR-T cells (2 million) and IL-15 after 5 days in vivo. FIG. 22A-FIG. 22C shows images of Hematoxylin and Eosin staining under various magnifications (indicated by scale bars), where the highest magnification image shows cells in the center of the hydrogel with signs of significant matrix deposition. FIG. 22D-FIG. 22F show images of CD3 staining (pink) under various magnifications (indicated by scale bars).

FIG. 23A-FIG. 23F shows results of levels of inflammatory mouse cytokines measured in the blood 3 days after treatment with CAR-T cells (2 million) in various delivery methods reported relative to values determined for naive mice (n=3 mice per group). Low IL-15 groups received 0.25 μg/mouse IL-15 (equivalent dose to efficacy studies). High IL-15 groups received 2.5 μg/mouse IL-15. FIG. 23A shows results for TNFa. FIG. 23B shows results for IL-6. FIG. 23C shows results for IL-1B. FIG. 23D shows results for GM-CSF. FIG. 23E shows results for IFNg. FIG. 23F shows results for IL-10.

FIG. 24A-FIG. 24F show levels of inflammatory human cytokines measured in the blood 3 days after treatment with CAR-T cells (2 million) in various delivery methods reported relative to values determined for naive mice (n=3 mice per group). Low IL-15 groups received 0.25 μg/mouse IL-15 (equivalent dose to efficacy studies). High IL-15 groups received 2.5 μg/mouse IL-15. FIG. 24A shows results for TNFa. FIG. 24B shows results for IL-6. FIG. 24C shows results for IL-1B. FIG. 24D shows results for GM-CSF. FIG. 24E shows results for IFNg. FIG. 24F shows results for IL-10.

FIG. 25A-FIG. 25C show results from analysis of expression of T cell activation markers on CAR-T cells extracted from PNP hydrogels. FIG. 25A shows MFI of PD1 staining. FIG. 25B shows MFI of 4-1BB staining. FIG. 25C shows PDI of CD39 staining. The top panels indicate CD4+ PDI and the bottom panels indicate CD8+ PDI. CAR-T cells were collected 10 days after treatment in the MED8A tumor model. Data shown as mean±SEM (n=3).

FIG. 26A-FIG. 26D shows results from T cell memory subsets analysis from CAR-T cell treated mice. T cell memory subsets, as determined by CD62L and CD45RA staining, from FIG. 26A (blood CD8+ cells) and FIG. 26B (blood CD4+ cells) show results from blood samples. FIG. 27C (spleen CD8+ cells) and FIG. 27D (spleen CD4+ cells) show results from spleen samples. Samples were collected 10 days after CAR-T cell administration in the MED8A tumor model. Data shown as mean±SEM (n=3). In the results, from top to bottom are EM, TCM, TSCM, and EF/EMRA.

FIG. 27A and FIG. 27B show results from T cell counts from CAR-T cell treated mice. Quantification of T cells from FIG. 27A (blood samples) and FIG. 27B (spleen samples) collected 10 days after T cell administration in the MED8A tumor model. Data shown as mean±SEM (n=3).

FIG. 28 shows the proportion of mice cured over the course of the experiment (n=5 mice for both groups). For these studies, “cured” was defined as exhibiting a luminescent tumor signal dropping below, and staying below, the background signal of the in vivo imaging apparatus.

FIG. 29A and FIG. 29B shows results from an in vivo experiment comparing CAR-T cell expansion when administered subcutaneously on the contralateral (left) flank, distal to the tumor (right subcutaneous flank). CAR-T cells (2 million) were co-administered in PNP-1-5 hydrogels or in a saline bolus at a dose of 0.25 μg/mouse IL-15. FIG. 29A shows CAR-T cell imaging using an in vivo imaging system (n=5 for all groups). FIG. 29B shows corresponding quantification of luminescent signal from CAR-T cell imaging (n=5 for all groups).

FIG. 30A-30E show CAR-T cell counts and memory subsets from distally treated mice. FIG. 30A shows results of quantification of total CAR-T cells from blood. CAR-T cell memory subsets, as determined by CD62L and CD45RA staining, from blood samples (FIG. 30B and FIG. 30C) and spleen samples (FIG. 30D and FIG. 30E). CAR-T cells were collected 10 days after treatment in the MED8A tumor model. Data shown as mean±SEM (n=3). Results from top to bottom are EM, TCM, TSCM, and EF/EMRA.

### DETAILED DESCRIPTION

Adoptive cell therapy (ACT) is a promising strategy to treat cancer. In the ACT process, immune cells are collected from a patient, isolated and engineered with receptors to combat cancer, expanded and then infused into the patient as the treatment. Chimeric antigen receptor (CAR) T cells are engineered to target an antigen that is over-expressed on cancer cells. This strategy has been widely effective in treating blood cancers, including B-cell leukemias and lymphomas, but has previously seen limited success in treating solid tumors. Several therapies for treating B-cell malignancies have recently been approved by the United States Food & Drug Administration (US FDA), and numerous concerted efforts are underway to translate this success to solid tumor applications. Unfortunately, large numbers of CAR-T cells are typically required for current treatment strategies to be successful, which requires costly, complex, and labor intensive ex vivo expansion of cells for 7-10 days. Moreover, extended ex vivo expansion can often limit the effector potential of transferred CAR-T cells. Thus, strategies for reducing the ex vivo expansion time by decreasing the required CAR-T cell dose would help in the clinical translation and broad dissemination of CAR-T therapy. Additionally, reducing the dose of cells needed for treatment would likely reduce the severe side effects associated with treatment, including as cytokine storm and neurotoxicity.

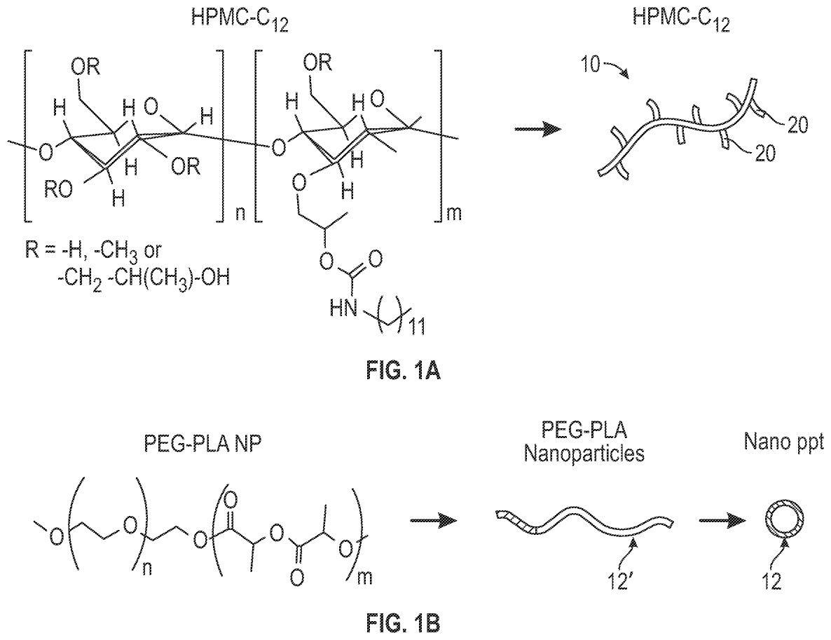

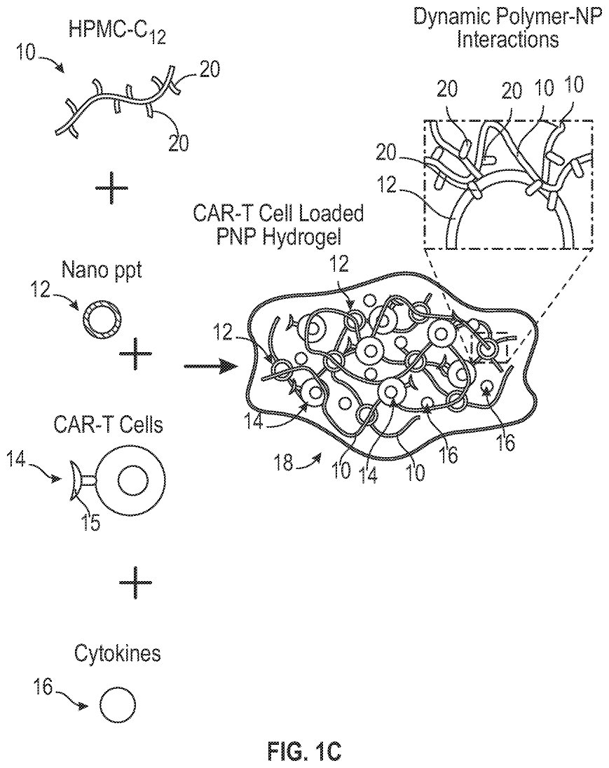

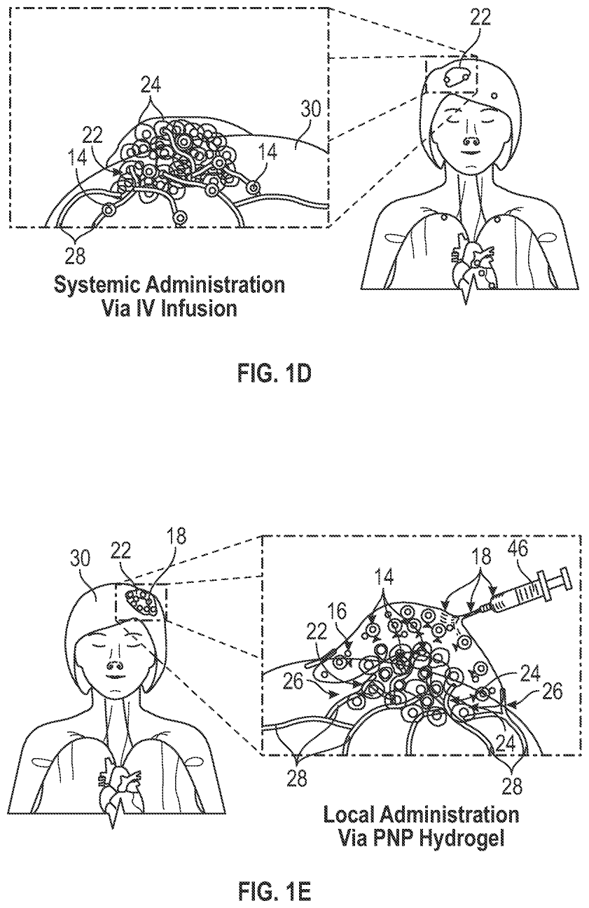

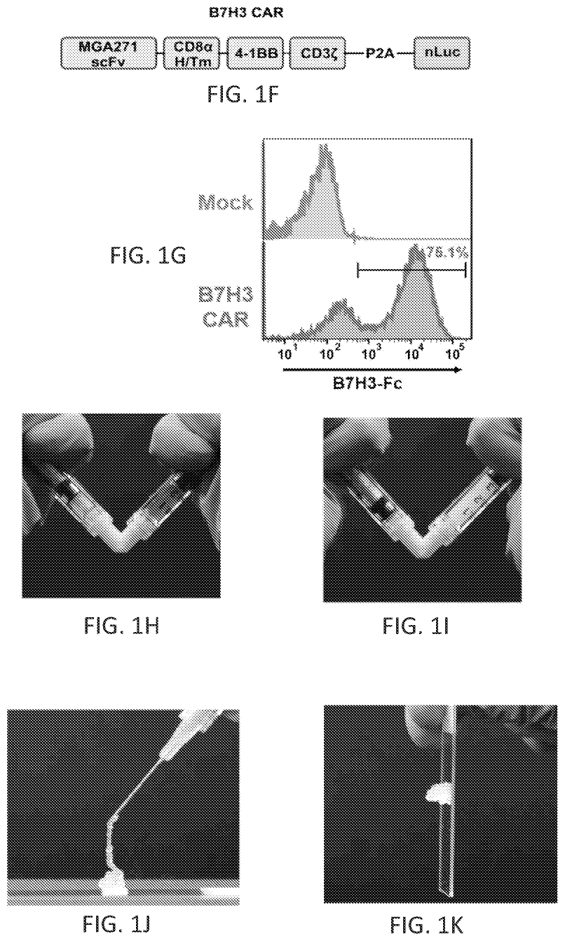

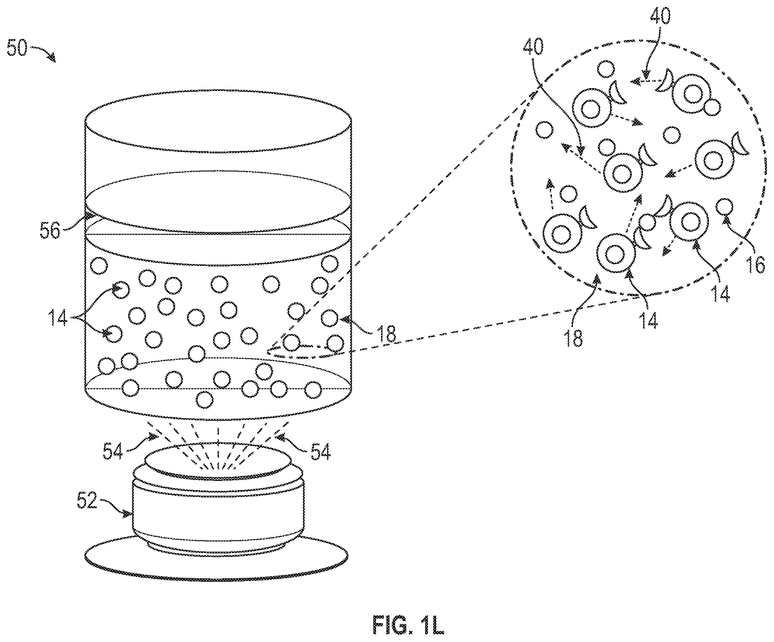

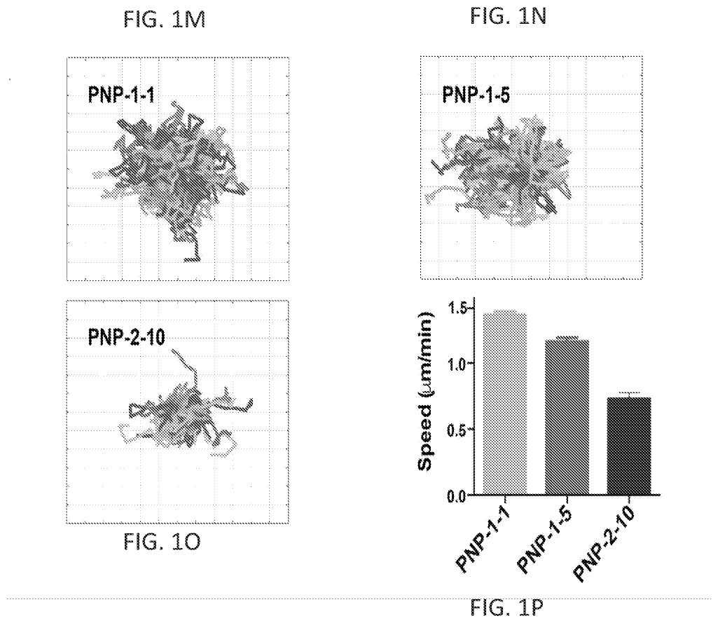

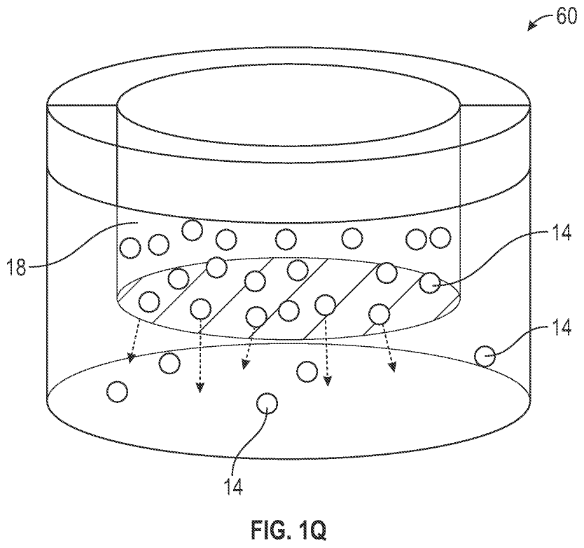

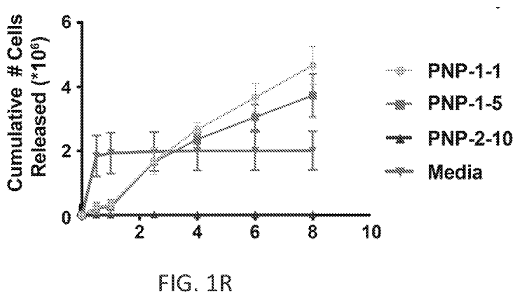

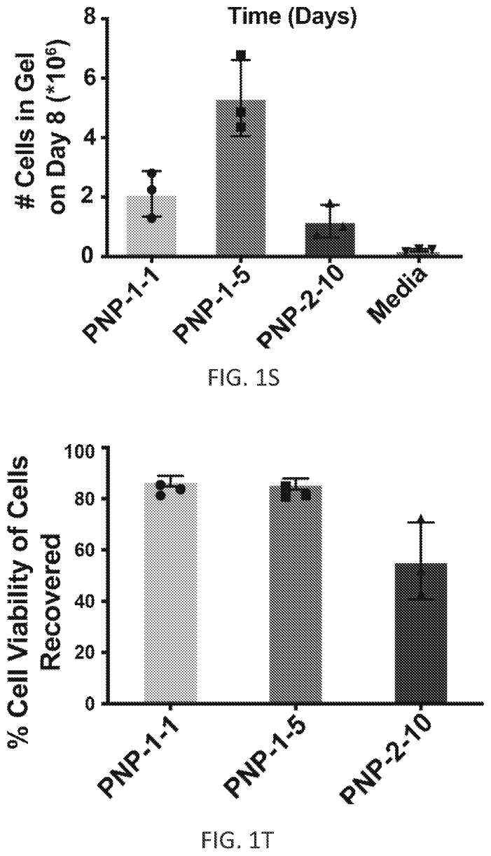

Currently, CAR-T cells are delivered through intravenous (IV) infusion through blood vessel 28, such as shown in FIG. 1D, which is effective in treating blood cancers because the T cells do not need to find and penetrate a solid tumor microenviroment. Unfortunately, T cells 14 administered in this way often become trapped in the lungs and exhibit poor infiltration of solid tumors. Locoregional cell delivery methods, particularly those exploiting biomaterial scaffolds, have shown improved local expansion of T cells at the tumor site, improving tumor infiltration and enhancing treatment of solid tumors. Unfortunately, the biomaterials scaffolds developed for locoregional ACT have required invasive surgical implantation procedures to reach tumor sites, hindering translation.

CAR-T cells may be most effective when fully activated state at the tumor site. The high local cytokine concentrations required for certain CAR-T activation are highly toxic if delivered systemically. Currently, T cells are expanded in high concentrations of cytokines only prior to delivery to avoid these toxicities. Locoregional approaches for delivering cytokines limit systemic cytokine exposure and reduce toxicities, and as such exhibit the potential to maintain adoptive T cells in a highly activated state in vivo.

Due to these promising results from locoregional studies for both the delivery of CAR-T cells and cytokines, these next generation biomaterials may transform treatment strategies for the controlled co-delivery of CAR-T cells and stimulatory molecules. Described herein are systems, methods, and compositions (including kits) utilizing a hydrogel useful for encapsulating and delivering cells and other cargo. The cells and other cargo may be immunomodulatory components delivered to a patient in need of therapy of immunotherapy.

### Definitions

Adoptive cell transfer refers to immune cells transferred into a patient. Adoptive cells may be isolated from the patient or from another subject, genetically modified, passaged (expanded) in vitro, and transferred into a patient. Adoptive cells that can be transferred include lymphocytes (T cells), natural killer cells, dendritic cells, and stem cells.

Natural killer (NK) cells refers to cells of the immune system that normally kill target cells in the absence of a specific antigenic stimulus, and without restriction according to major histocompatibility complex (MHC) class. Target cells may be cancer or tumor cells. Natural killer cells have historically been characterized by the presence of CD56 and the absence of CD3 surface markers and other forms of natural killer cells have also been characterized. Natural killer cells are generally heterogeneous populations of cells within which natural killer cells have been enriched. Endogenous NK cells may be intended for autologous or allogeneic treatment of a patient.

Autologous refers to cells that come from the same person who has or will receive the cells. As T cells are genetically matched to an individual, transferring autologous T cells reduces risk of graft versus host disease (GvHD) in a person receiving cells.

Allogeneic refers to cells that come from a different donor than the recipient who will receive the cells. As T cells are genetically matched to an individual, allogeneic T cell transfer can cause very serious complications, referred to as graft-versus-host-disease (GVHD). T cells (e.g., CAR-T cells) can be subject to a complex gene-editing process to reduce the risk of GVHD during allogeneic cell transfer. While T cells are genetically matched to individuals, natural killer cells are not. As such, a donor's natural killer cells can be injected into a recipient without concern about graft-versus-host-disease (GvHD). That also means allogeneic CAR natural killer cells don't need to undergo the complex gene-editing process that allogeneic CAR-T cells do.

Graft-versus-host-disease (GVHD) refers to a potentially serious complication of certain allogeneic cell transfers. Graft-versus-host-disease can be mild, moderate, severe, or life-threatening. The recipient's body views the host cells as foreign and attacks them, leading to a range of effects on the skin, the gastrointestinal tract, or the liver, such as rashes, blisters, nausea, vomiting, abdominal cramps, loss of appetite, diarrhea, liver damage, and jaundice.

Cytokine refers to a general class of biological molecules important in cell signaling in the immune system. Cytokines were originally identified as small proteins of about 5 kDa to about 20 kDa in size secreted by specific cells of the immune system. A cytokine acts through its own receptor on target cells, and these receptors include members of the immunoglobulin (Ig) superfamily and tumour necrosis factor (TNF). Cytokine is a general name; other names are defined based on their presumed function, cell of secretion, or target of action. For example, cytokines made by lymphocytes can also be referred to as lymphokines. Many of the lymphokines are also known as interleukins (ILs), since they are not only secreted by leukocytes but also able to affect the cellular responses of leukocytes. Those cytokines secreted by monocytes or macrophages are termed monokines. And chemokines are cytokines with chemotactic activities. Examples of cytokines include IL-2, IL-12, IL-15, IL-18 and IL-21.

Integrin refers to a transmembrane cell surface receptor for binding to extracellular ligands, cell-ligands, and soluble ligands. Integrins have been characterized as heterodimeric proteins with an α subunit and a β subunit, with the α subunit and a β subunit having distinct domain structures. Different α subunit and β subunits may heterodimerize with, respectively different β and α subunits. For example, β1 may (separately) heterodimerize with α1, α2, α3, and α4. Integrins of interest include those binding arginine-glycine-aspartic acid (RGD).

Physical crosslink refers to crosslinks and can include entangled chains, hydrogen bonding, hydrophobic interactions, and crystallite formation in a polymer.

Solid tumor refers to an abnormal, solid mass of cells. The mass is devoid of fluids or cysts. Solid tumors include sarcomas, such as tumors occurring in blood vessels, bone, fat tissue, ligaments, lymph vessels, muscles, or tendons and carcinomas, such as tumors occurring in epithelial cells, including in the skin, glands, and lining of organs.

Intratumoral refers to within a tumor.

Immunotherapy refers to the treatment or prevention of a disease or condition by a method comprising inducing, enhancing, suppressing or otherwise modifying an immune response.

Chimeric antigen receptor (CAR) refers to an artificially constructed hybrid protein or polypeptide with an extracellular antigen-binding domain that is fused to an intracellular signaling domain. The extracellular antigen-binding domain can be an antigen binding domain of an antibody (e.g., a single chain variable fragment (scFv)) that is specific for an antigen. An scFv domain can be engineered to recognize any antigen, including tumor-specific antigens. An intracellular signaling domain can be linked to cell signaling or cell activation domains. CARs have the ability to redirect cell specificity and reactivity toward a selected target in a non-MHC-restricted manner, exploiting the antigen-binding properties of monoclonal antibodies. The non-MHC-restricted antigen recognition gives cells expressing CARs the ability to recognize an antigen independent of antigen processing, thus bypassing a major mechanism of tumor escape. CARs of interest are expressed in T cells or natural killer (NK) cells to increase cytotoxicity.

Tumor specific antigen refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell. With cancer, phenotypic changes distinguishing a tumor cell from normal cells derived from the same tissue are often associated with one or more changes in the expression of specific gene products, including the loss of normal cell surface components or the gain of others (i.e., antigens not detectable in corresponding normal, non-cancerous tissue). Tumor-specific antigens may serve as markers for tumor phenotype. Examples of tumor specific antigens include those assigned to three main groups: cancer/testis-specific antigen (e.g. MAGE, BAGE, GAGE, PRAME and NY-ESO-1), melanocyte differentiation antigens (e.g. tyrosinase, Melan-A/MART, gp100, TRP-1 and TRP-2) and mutated or aberrantly expressed antigens (e.g. MUM-1, CDK4, beta-catenin, gp100-in4, p15 and N-acetylglucosaminyltransferase V).

A drug refers to a medicine or other substance which has a physiological effect when introduced into the body.

Effective amount refers to a quantity of a composition or material sufficient to achieve a desired therapeutic effect, e.g., an amount which results in the amelioration of the cancer cells or one or more symptoms associated with a disease (e.g., cancer). The amount of first immunomodulatory cargo (e.g., cells) or second immunomodulatory cargo (e.g., cytokine) administered to a subject can depend on the type and progression of the cancer and on the characteristics of the individual, such as general health, age, sex, body weight and tolerance to drugs. It can also depend on the degree, severity and type of disease. The skilled artisan will be able to determine appropriate dosages depending on these and other factors. Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

Isolating a cell refers to a process of dissociating or otherwise removing a cell from a tissue sample (e.g., blood tissue, placental tissue), and separating the cell from other cells or non-cells in the tissue. Isolated cells will generally be free from contamination by other cell types and will generally be able to be propagated and expanded.

An isolated cell e.g., an isolated T cell, includes a cell that is substantially separated from other, different cells of the tissue, e.g., blood or placenta from which the cell is derived. A cell is “isolated” if at least 50%, 60%, 70%, 80%, 90%, 95%, or at least 99% of the cells with which the population of cells, or cells from which the population of cells is derived, is naturally associated, i.e., cells displaying a different marker profile, are removed from the cell, e.g., during collection and/or culture of the cell. In some embodiments, an isolated cell exists in the presence of a small fraction of other cell types that do not interfere with the utilization of the cell for analysis, production or expansion of the cells. A population of isolated cells can be at least 30%, 40%, 50%, 60%, 70%, 80%, 85%, 90%, 95%, 98%, or 99% pure, or any interval thereof. In a specific embodiment, a population of isolated cells are at least 98% or at least 99% pure. As used herein, the term “population of isolated cells” means a population of cells that is substantially separated from other cells of a tissue, e.g., blood, from which the population of cells is derived.

### DESCRIPTION

Described herein is an immunotherapy delivery system with an injectable and self-healing polymer-nanoparticle (PNP) hydrogel to deliver a first immunomodulatory cargo, such as cells, and an immunomodulatory cargo, such as a cell modulator. Polymer-nanoparticle hydrogels are a type of supramolecular hydrogel where the polymeric constituents are held together by dynamic non-covalent interactions between polymers and nanoparticles. Such polymer-nanoparticle hydrogels can benefit from many of the favorable characteristics of traditional covalently cross-linked hydrogels, such as high drug loading capacity, gentle conditions for encapsulation of biologic cargo, sustained delivery of cargo, and mechanical tunability. In addition, unlike traditional covalently cross-linked hydrogels, polymer-nanoparticle hydrogels can be easily administered as immunotherapy due to their shear thinning and self-healing properties. Sheer thinning includes materials (e.g., fluids) whose viscosity depends on shear rate (e.g., decreases under shear strain). The fabrication process for the polymer-nanoparticle hydrogels described herein, which is scalable and therefore highly translatable, involves simple mixing of the polymer, nanoparticles (NPs), an aqueous solution of the first immunomodulatory cargo and the second immunomodulatory cargo. FIG. 1A-FIG. 1C schematically illustrate an example of polymer-nanoparticle (PNP) hydrogel. The polymer-nanoparticle (PNP) hydrogel includes a polymer, such as HPMC-C₁₂ 10 shown in FIG. 1A, a nanoparticle, such as PEG-PLA NP 12 shown in FIG. 1B, CAR-T cells 14, and cytokines 16 as shown in FIG. 1C. FIG. 1C also shows PNP hydrogel 18 loaded with CAR-T cells 14 and cytokines 16. The PNP hydrogel 18 contains crosslinks 20 that create a mesh-like hydrogel structure. The mesh-like hydrogel structure contains and holds the CAR-T cells 14.

The polymer-nanoparticle (PNP) hydrogel immunotherapy delivery system described herein includes a polymer non-covalently crossed-linked with a plurality of nanoparticles, a cell adhesion motif in the hydrogel configured to reversibly adhere to and release cells, a first immunomodulatory cargo comprising cells encapsulated in the hydrogel wherein at least a portion of the cells are adhered to the cell adhesion motif, and a second immunomodulatory cargo encapsulated in the hydrogel.

The cell adhesion motif may be a peptide configured to reversibly adhere to and release the cells. Some cells may be adhered to the hydrogel by the cell adhesion motif and then released from the hydrogel and travel in the body, such as to or into a solid tumor. Cells may travel through the hydrogel by successively adhering to and being released from a plurality of cell adhesion motifs. Cell adhesion motifs may act as “handholds” for cells. Attraction between a cell and the cell adhesion motif may “pull” a cell through the hydrogel, and this process may be repeated a plurality of times. A cell may thus move through the hydrogel without requiring covalent bonds to be broken. The cell adhesion motif may reversibly adhere and release a plurality of different cells. The cell adhesion motif may be specific or generic. The cell adhesion motif may be configured to bind to a binding partner on the cells. For example, the cell adhesion motif may bind integrins (e.g., specifically bind integrins) or other extracellular matrix receptors on the cells. The cell adhesion motif may include a part or entirety of a cell binding protein. For example, the cell adhesion motif may include part or all of fibronectin, vitronectin, or another extracellular matrix molecule.

An exemplary cell adhesion motif is arginine-glycine-aspartic acid (RGD) peptide, a cell adhesion motif found in the extracellular matrix glycoprotein fibronectin. T cells and natural killer (NK) cells express integrins and arginine-glycine-aspartic acid (RGD) peptide is recognized by the integrins. Other cell adhesion motifs and binding partners can also be used. For example, cells can be genetically modified to express a binding partner to a cell adhesion motif attached to a hydrogel.

The cell adhesion motif may be covalently or non-covalently attached to a hydrogel. In some examples, a cell adhesion motif is covalently attached to a nanoparticle. In some examples, a nanoparticle presents a cell adhesion motif. A cell adhesion motif may be located on an outside of a nanoparticle. An arginine-glycine-aspartic acid (RGD) peptide can be attached to a nanoparticle polymer, such as to a poly(ethylene glycol)-bpoly(lactic acid) molecule on a nanoparticle.

FIGS. 1Q-1T illustrate that the PNP system described herein can release a controlled amount of CAR-T cells over time. FIG. 1Q schematically illustrates testing device 60 for determining cell release over time. CAR-T cells are loaded into hydrogel formulation 18 and the hydrogel formulation is placed in holder in testing device 60. Cells travel through hydrogel formulation 18 and exit the bottom of the holder, as shown by the arrows, where they are counted.

In some variations, an immunotherapy delivery system cell adhesion motif may include a plurality of different types of cell adhesion motifs.

In some examples, an immunotherapy delivery system may include nanoparticles, wherein the polymers in the nanoparticles have a ratio between 10:90 and a 90:10 or a ratio between 25:75 and 75:25 of polymer with a cell adhesion motif and polymer without a cell adhesion motif. In some particular examples, an immunotherapy delivery system may include polymers in nanoparticles comprising between a 10:90 and a 90:10 ratio of poly(ethylene glycol)-bpoly(lactic acid) (PEG-PLA) with the cell adhesion motif to poly(ethylene glycol)-bpoly(lactic acid) (PEG-PLA) without the cell adhesion motif. In some examples, nanoparticles comprise between a 25:75 ratio and a 75:25 ratio of poly(ethylene glycol)-bpoly(lactic acid) (PEG-PLA) with the cell adhesion motif to poly(ethylene glycol)-bpoly(lactic acid) (PEG-PLA) without the cell adhesion motif. As described above, the PNP hydrogel immunotherapy delivery system described herein can include a first immunomodulatory cargo including cells. The first immunomodulatory cargo can be reversibly bound to the hydrogel. For example, cells can be reversibly (e.g., non-covalently) bound to the cell adhesion motif. In some embodiments, the cells are adoptive cells of the immune system, such as T cells or natural killer cells. Cells of interest can be genetically modified, such as to express a chimeric antigen receptor (CAR) on its surface.

As also described above, the PNP hydrogel immunotherapy delivery system described herein can include a second (third, fourth, etc.) immunomodulatory cargo. The second immunomodulatory cargo can be a peptide/protein or can be a component other than a peptide/protein. The second immunomodulatory cargo may be configured to act on another component in the hydrogel, such as to act on cells encapsulated in the hydrogel. The immunomodulatory cargo may be configured to modulate the cells, such as by activating them. The immunomodulatory cargo can be, for example, cytokines, such as immune stimulatory cytokines. Cytokines in the hydrogel may act to stimulate and/or expand cells in the hydrogel. For example, the cytokines can stimulate immune cells in the hydrogel, such as T cells or natural killer. As the cells grow and divide in hydrogel, available cytokine can activate the cells in the hydrogel, such as prior to cell release into a patient's body.

The immunomodulatory cargo, such as cytokines, can aid in the PNP hydrogel immunotherapy delivery system behaving as a cell or immune niche or depot, stimulating immune cells which can be released from the hydrogel over a period of hours, days, weeks, or months, such as for at least one day, at least two days, at least three days, at least four days, at least five days, at least six days, at least a week, at least two weeks, at least three weeks, or at least four weeks. Immunotherapy can be delivered from the PNP hydrogel immunotherapy delivery system over these time periods, such as for two to three weeks.

The PNP hydrogel immunotherapy delivery system may be configured to prevent substantial migration of a second (third, fourth, etc.) immunomodulatory cargo, such as a cytokine. For example, physical crosslinks in the hydrogel may prevent the second immunomodulatory cargo, such as a cytokine, from diffusing through the hydrogel or from substantially being released from the hydrogel. Thus, in some examples, no covalent bonds need to be broken in the hydrogel to activate a first immunomodulatory cargo, such as cells, with a second immunomodulatory cargo, such as a cytokine. Advantageously, the PNP hydrogel, as it continues to encapsulate second immunomodulatory cargo (such as cytokines) can shield a patient's body from the cargo, preventing toxicity while delivering a sufficiently high dose of the cargo to the immunotherapy cargo (such as cells). In some variations, a second (or additional) immunomodulatory cargo may be configured to act on a substance outside of the hydrogel instead of or in addition to being configured to act on another component in the hydrogel. For example, an additional immunomodulatory cargo may include an effective dose of an active agent, such as a drug.

The shear-thinning and self-healing polymer nanoparticle hydrogels described herein have favorable material properties and a mild synthesis approach that is scalable and versatile due to their ability to load biologic cargo by simple mixing, making them well-suited for encapsulation and delivery of adoptive cells. The cargo-loaded hydrogels are injectable and can retain their solid-like structure when under low stresses, enabling creation of a new stimulatory microenvironment within the body and sustained delivery of immunotherapies. The polymer nanoparticle hydrogel can be used, for example, to replace phosphate buffered saline (PBS) as a delivery vehicle and can be used with any immunotherapy. The unique dynamic network rearrangement in these materials enables the surprising release of larger cargo and retention of smaller cargo and the larger and small cargo can be dramatically different in size or chemical makeup.

In some embodiments, the polymer nanoparticle hydrogels described herein can be made of one or more polymers, such as cellulose derivatives, such as hydroxypropylmethylcellulose (HPMC), hydroxyethylcellulose (HEC), methylcellulose (MC), carboxymethylcellulose (CMC), or hydroxypropylcellulose (HPC), or hyaluronic acid (HA) optionally modified with a hydrophobic moiety, such as hexyl (-C6), octyl (-C8), deceyl (-C10), dodecyl (-C12), phenyl (Ph), adamantyl, tetradecyl (-C14), oleyl, or cholesterol (e.g., 5-30% modification, such as 5-25% modification, such as approximately 10-15% or 25%). In one specific embodiment, HPMC is 10-15% modified with dodecyl. In another specific embodiment, HEC is 25% modified with dodecyl. In another specific embodiment, HEC is 10% modified with cholesterol. Further, the polymer can be mixed with nanoparticles, such as nanoparticles having a diameter of less than 100 nm, such as 30-50 nm, such as approximately 40 nm. The nanoparticles can be core-shell nanoparticles with hydrophobic cores, such as poly(ethylene glycol)-block-poly(lactic acid) (PEG-PLA) or poly(ethyleneglycol)-block-poly(caprolactone) (PEG-PCL) nanoparticles.

Further, in some embodiments, the PNP hydrogels can be made of greater than 1% polymer by weight, such as greater than 1% and less than 5% by weight, such as 1.5-3% polymer by weight, such as approximately 2% polymer by weight. In some embodiments, the PNP hydrogel can include 4-12% nanoparticles, such as 8-11%, such as 10% nanoparticles. Having a percentage of nanoparticles within these ranges helps ensure that the PNP remains stable. As used here, an “X:Y gel” can refer to X wt % polymer and Y wt % nanoparticles. The PNP hydrogel described herein is configured to dissolve through the noncovalent bonds. The PNP hydrogel described herein can include additionally or alternatively include any of the characteristics and/or features of the hydrogels described in U.S. Publication No. 2017/0319506 and WO Publication No. 2020/072495, the entirety of which are incorporated by reference herein.

Also described herein is a method of treating a disease including delivering any of the PNP hydrogel immunotherapy delivery systems described herein to a patient and releasing the cells from the hydrogel. FIG. 1E schematically illustrates delivering a PNP hydrogel immunotherapy delivery systems to a tumor 22 of a brain 30 of a patient. FIG. 1E illustrates the PNP hydrogel 18 being delivered using a delivery device 46, such as a syringe. The PNP hydrogel 18 is delivered next to the tumor 22. In some variations, methods include PNP hydrogel delivering PNP hydrogel (and associated CAR-T cells 14) into the tumor, on top of the tumor, around part or all of the tumor, or remote from the tumor. The PNP hydrogel 18 releases the CAR-T cells 18 (the CAR-T cells 18 crawl out of the PNP hydrogel 18). CAR-T cell receptor 15 binds to antigen 26 on tumor cell 24. The method may further include releasing the cells from the hydrogel for a period of time ranging from one day to four weeks, or for at least one day, at least two days, at least three days, at least four days, at least five days, at least six days, at least a week, at least two weeks, at least three weeks, or at least four weeks or any time between these. Any of the methods described herein may include activating the cells with the immunomodulatory cargo. Any of the methods described herein may include expanding the number of cells in the hydrogel such as by 2-fold, 3-fold, 5-fold, 10-fold, or 100-fold. Any of the methods described herein may include treating cancer and/or a solid tumor. Any of the methods described herein may include releasing adoptive cells, such as T cells or natural killer cells. Any of the methods described herein may include releasing cells that express a chimeric antigen receptor (CAR) that recognizes a tumor antigen. Any of the methods described herein may include releasing allogeneic cells or autologous cells. Any of the methods described herein may further include further removing the cells from the patient or from a donor; isolating the removed cells; expanding the number of cells in vitro; and encapsulating the cells in the hydrogel prior to the delivering step. Any of the methods described herein may include delivering the immunotherapy delivery system through a syringe or catheter. Any of the methods described herein may include delivering the immunotherapy delivery system to the patient by a route selected from the group consisting of intravenous, intraperitoneal, intramuscular, intratumoral, and subcutaneous. An immunotherapy delivery system may be delivered to a patient locally to a region of the patient needing treatment. A region of a patient needing treatment may be a cancerous region, a mole, a polyp, a solid cancer tumor, a benign growth, or a non-benign growth, a cancerous region, or a precancerous region. In some examples, the immunotherapy delivery system may be delivered to a solid tumor cancer (brain tumor, breast tumor, colon tumor, etc.) in the patient, such as by injection into the solid tumor cancer. A solid tumor cancer may be detected by biopsy (e.g., bone marrow biopsy, endoscopic biopsy, excisional or incisional biopsy, fine needle aspiration biopsy, punch biopsy, shave biopsy, skin biopsy), endoscopy (e.g. cystoscopy, colonoscopy, sigmoidoscopy), imaging (such as transmission imaging (e.g., X-rays, computed tomography (CT) or computed axial tomography (CAT) scan, fluoroscopy), reflection imaging (e.g., ultrasound), or emission imaging (e.g., magnetic resonance imaging (MRI)), palpation, or surgery.

In this and other methods, delivering includes delivering the immunotherapy delivery system to the patient. In this and other methods, delivering includes delivering the system to one or more than one location remote from a region of the patient needing treatment. A region of a patient needing treatment may be a cancerous region, a mole, a polyp, a solid cancer tumor, a benign growth, or a non-benign growth, a cancerous region, or a precancerous region. In this and other methods, the disease includes a solid tumor cancer, and delivering the immunotherapy delivery system to the patient includes delivering the system to a location in the patient remote from the solid tumor cancer. As indicated above, delivering the system to a location remote from a region of a patient needing treatment can include delivering by a route such as intravenous, intraperitoneal, intramuscular, intratumoral, and subcutaneous. In some examples, delivering the immunotherapy delivery system to a patient may include delivery to one location, multiple locations, and/or systemic delivery. If the immunotherapy is delivered to more than one location, the locations may be local, remote, or both local and remote (immunotherapy may be delivered to one or more local locations and one or more remote locations).

Also described herein are kits that may be useful for immunotherapy delivery. A kit may include a polymer precursor, a nanoparticle precursor, and/or a cell adhesion motif. Some kits may include an immunomodulatory cargo. In some kits, the polymer precursor includes hydroxypropylmethylcellulose (HPMC) with hydrophobic lipid dodecyl chains. Some kits include a first polymer precursor including poly(ethylene glycol)-bpoly(lactic acid) (PEG-PLA) without a cell adhesion motif; and a second polymer precursor including poly(ethylene glycol)-bpoly(lactic acid) (PEG-PLA) with the cell adhesion motif. In some variations, the cell adhesion motif may be covalently or noncovalently attached to the second polymer precursor. In some variations, the immunomodulatory cargo includes a cytokine, such as an interleukin, such as IL-15.

Experimental:

In this hydrogel system, hydroxypropylmethylcellulose (HPMC) was modified with hydrophobic lipid dodecyl chains (C12) using isocyanate coupling chemistry. PEG-PLA nanoparticles (NPs) with a diameter of ˜40 nm were prepared using nanoprecipitation techniques, yielding core-shell nanoparticles (NPs) with a hydrophilic PEG-based corona and a hydrophobic PLA-based core. To promote cellular adhesion and viability, the cell adhesion motif arginine-glycine-aspartic acid (RGD) was attached to the hydrophilic end of the PEG-PLA copolymer through a copper-catalyzed “click” reaction prior to). A 25:75 physical mixture of RGD-functionalized PEG-PLA polymer (RGD-PEG-PLA) and unmodified PEG-PLA polymer was used to create the nanoparticles (NPs). Polymer-nanoparticle (PNP) hydrogels are formed by mixing aqueous solutions of the HPMC-C12 polymer and PEG-PLA NPs (FIG. 1A). Cells are easily suspended in the nanoparticle aqueous phase before mixing the nanoparticles (NPs) and polymer solutions. When these components are mixed, dynamic multivalent interactions between the hydrophobically-modified HPMC polymers and the surface of the PEG-PLA nanoparticles (NPs) cause physical crosslinking and hydrogel formation. Here we focus on a formulation containing 1 wt % HPMC-C12 and 5 wt % nanoparticles. The supramolecular polymer-nanoparticle (PNP) hydrogel platform exhibits shear-thinning behavior enabling injection through small diameter needles and protecting encapsulated cells from harsh mechanical forces in the syringe.

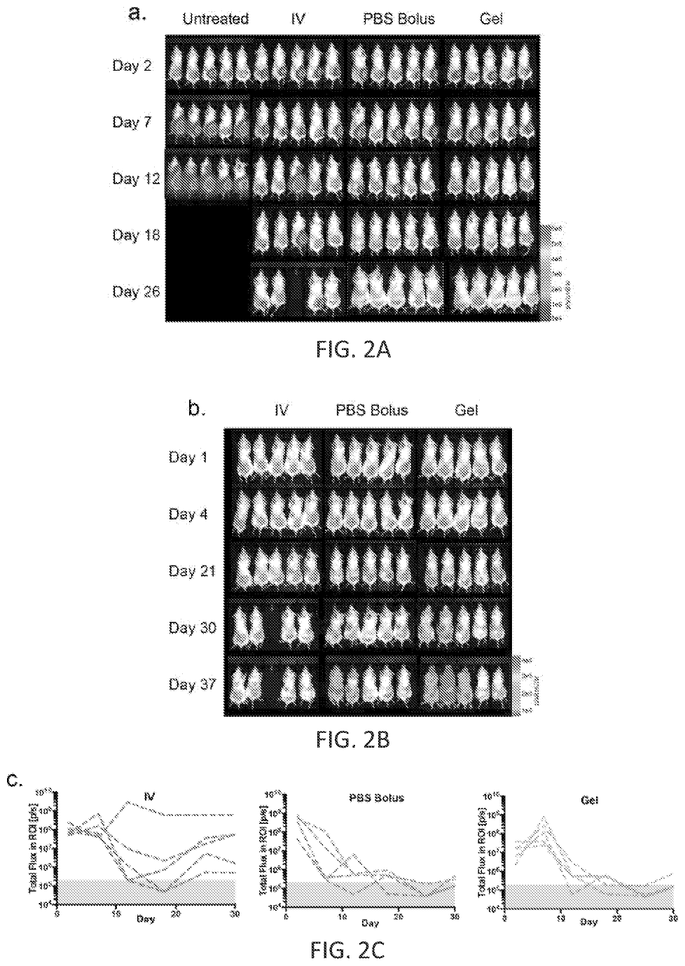

To investigate the effect of this treatment strategy to deliver CAR-T cells locally to tumor sites, a xenograft model of human medulloblastoma was implemented in NSG mice. This model was chosen as a relatively hard-to-treat case for intravenously delivered cells. B7H3 CAR 41BBZ CAR-T cells were expanded for 10 days before treatment. Tumors were engineered to express luciferase to enable luminescent monitoring of tumor size, while T cells were engineered to express Nano-luciferase. 8*10⁶ CAR-T cells were delivered per mouse with various treatment strategies, including traditional IV, a local saline (PBS) bolus injection, and a local injection of cells encapsulated in polymer-nanoparticle (PNP) hydrogels (100 uL injections). Tumors and T cells were monitored over time to assess treatment efficacy (FIG. 2A-FIG. 2C). At the end of the experiment, both the local PBS and polymer-nanoparticle (PNP) hydrogel strategies were successful in treating the tumors. The polymer-nanoparticle hydrogel group interestingly demonstrated very robust T cell expansion at the later time points. These results confirm that T cells can proliferate and migrate from polymer-nanoparticle (PNP) hydrogels to target tumors.

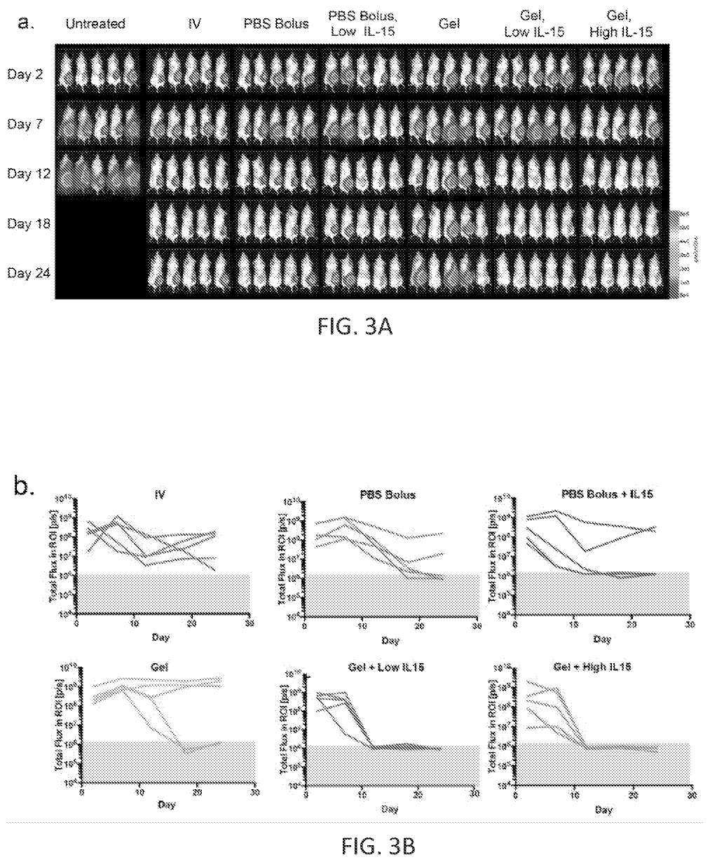

To further challenge the model, in the next study, a lower dose of 2*10⁶ cells were delivered per mouse with the various treatment strategies. In addition, 3 groups containing IL-15 were included in the study. IL-15 was added to the local PBS injection at one concentration (2.5 ug/mL) and the polymer-nanoparticle (PNP) hydrogel formulation at two different concentrations (2.5 ug/mL and 25 ug/mL). Tumors were monitored over time to assess treatment efficacy (FIG. 3A-FIG. 3B). At the end of the experiment, only the polymer-nanoparticle (PNP) hydrogel groups containing IL-15 completely eradicated the tumors. Due to the presence of tumors in the PBS group containing IL-15, the hydrogel likely locally retained IL-15 promoting local cell proliferation.

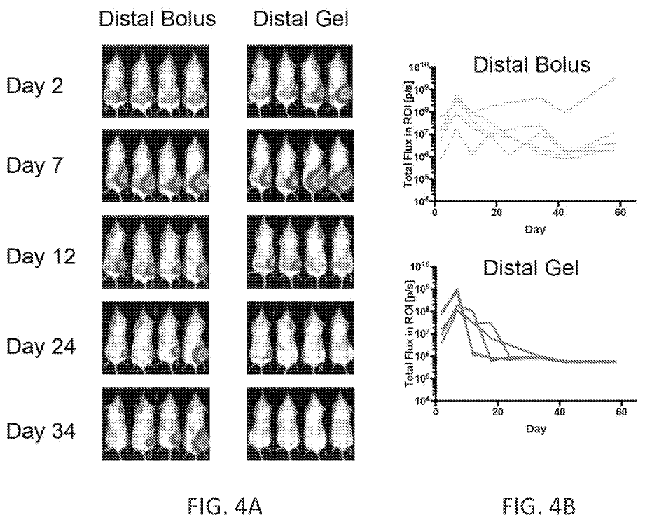

To further determine if polymer-nanoparticle (PNP) hydrogel immunotherapy could be used to treat tumors remote from the treatment site, polymer-nanoparticle (PNP) hydrogels with CAR-T cells were placed far from the tumor site. Hydrogels were prepared as described herein and delivered to the xenograft model of human medulloblastoma (e.g., as described in FIGS. 2A-2C), except that the polymer-nanoparticle (PNP) hydrogels with CAR-T cells were injected in the subcutaneous space on the opposite, distal side of the mouse from the tumor location. The tumor was measured over time through bioluminescence. Also, similar to as described above, a saline (PBS) bolus injection of CAR-T cells was performed (see left side of FIG. 4A and top of FIG. 4B). FIG. 4A and FIG. 4B show that mice that received the hydrogel treatment showed improved treatment relative to mice treated using a saline (PBS) bolus injection. Results from imaging the mice from day 2 to day 34 are shown in FIG. 4A. Bioluminescent imaging scale (y axis) on FIG. 4B is similar as shown in FIG. 2C and FIG. 3B and shows total flux in the region of interest (ROI) over time in days (x axis).

Overall, disclosed herein is a delivery strategy useful for cancer therapy. Advantageously, the PNP hydrogel immunotherapy delivery system described herein can be effectively used as a cancer therapy. In some embodiments, this hydrogel system uses minimal chemistry, enables rapid formulation, and allow for modular personalized incorporation of cytokines for treatment. In some examples, treatment may be performed with a simple injection at the tumor site. This method can be applied to CAR-T cells and other adoptive cell delivery applications such as delivering endogenous or modified natural killer cells.

To address these challenges, we engineer a biomaterials platform for CAR-T cell delivery based on injectable Polymer-Nanoparticle (PNP) hydrogels (FIG. 1A-1T). These hydrogels leverage highly scalable chemistries and can be formulated under mild conditions facilities facile encapsulation of therapeutic cells, while their injectability enables locoregional delivery of cells to tumors (FIG. 1C). To make these hydrogels, a solution of dodecylmodified hydroxypropylmethylcellulose (HPMC-C12; FIG. 1A) is mixed with a solution of biodegradable nanoparticles (NP) comprising poly(ethylene glycol)-b-poly(lactic acid) (PEG-PLA NPs). Upon mixing, dynamic multivalent interactions between the HPMC-C12 polymers and the PEG-PLA NPs cause physical crosslinking and formation of a robust hydrogel (FIG. 1C). PNP hydrogels retain their properties across a range of temperatures, their physical crosslinking enables facile injection through a needle or catheter, and can retain local signalling over prolonged and controlled timescales. To promote cellular motility and viability, the cell adhesion motif arginine-glycine-aspartic acid (RGD) was attached to the hydrophilic end of the PEG-PLA copolymers using a “click” reaction prior to nanoprecipitation. We have previously shown that cells can be easily suspended in the RGD-PEG-PLA NP solution prior to mixing with HPMC-C12.

There are inherent materials design challenges in both slowly delivering immunostimulatory molecules while engineering a matrix that will maintain T cell viability. While previously reported biomaterials have required conjugation of activating signals to materials scaffolds for either in vitro expansion and in vivo treatment, we have recently shown that the dynamic polymer mesh within PNP hydrogels can be engineered to be small enough to provide prolonged retention of proteins while still enabling cell motility. We hypothesize, therefore, that coencapsulation of immunostimulatory signals such as proliferative cytokines with CAR-T cells will enhance T cell expansion in vivo.

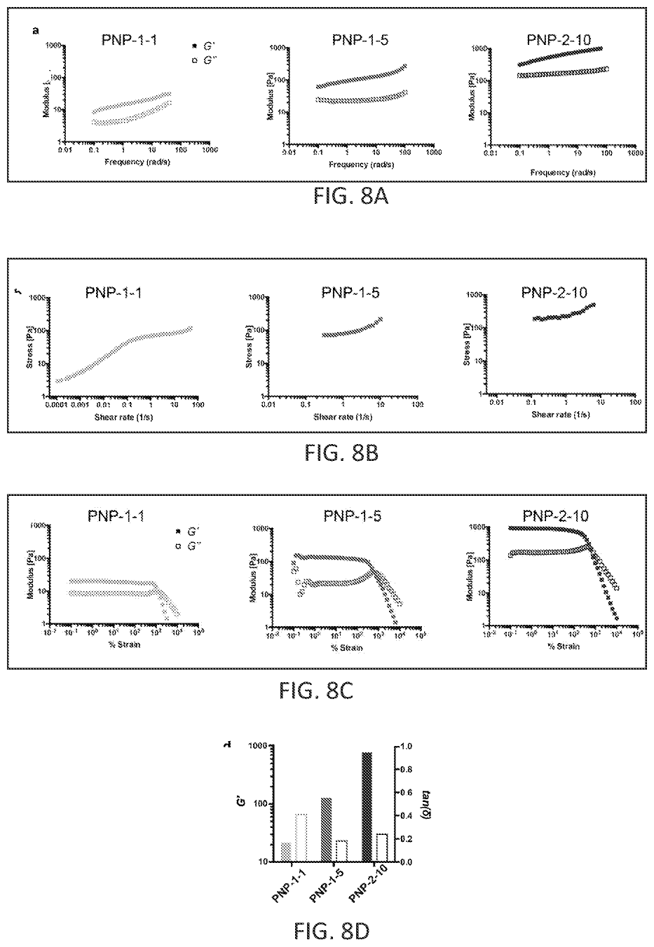

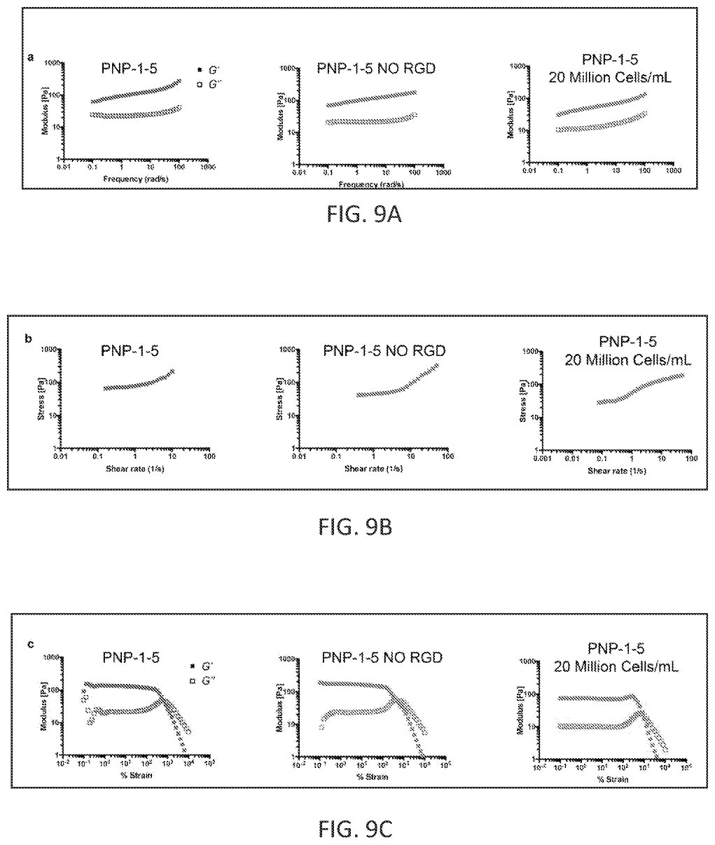

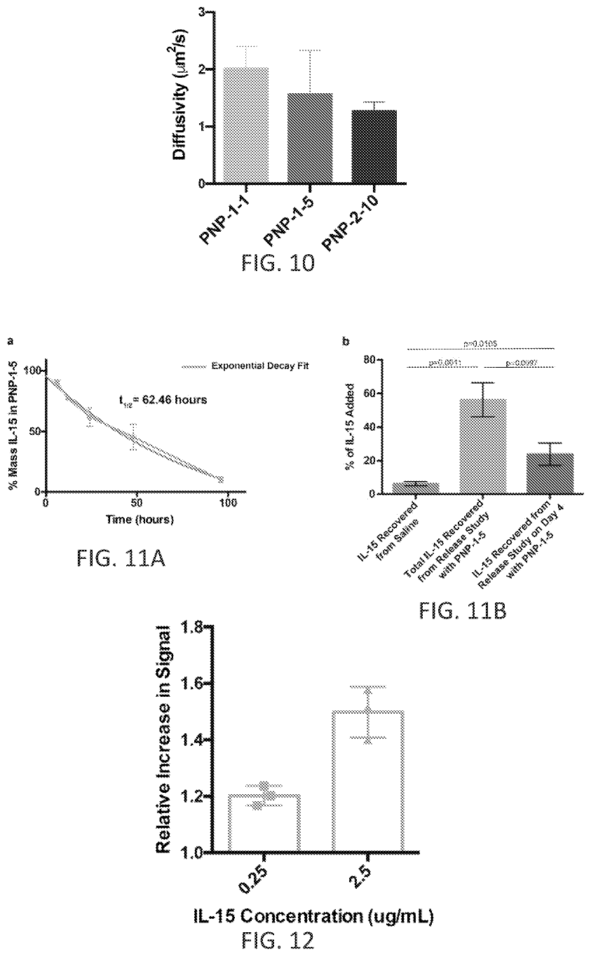

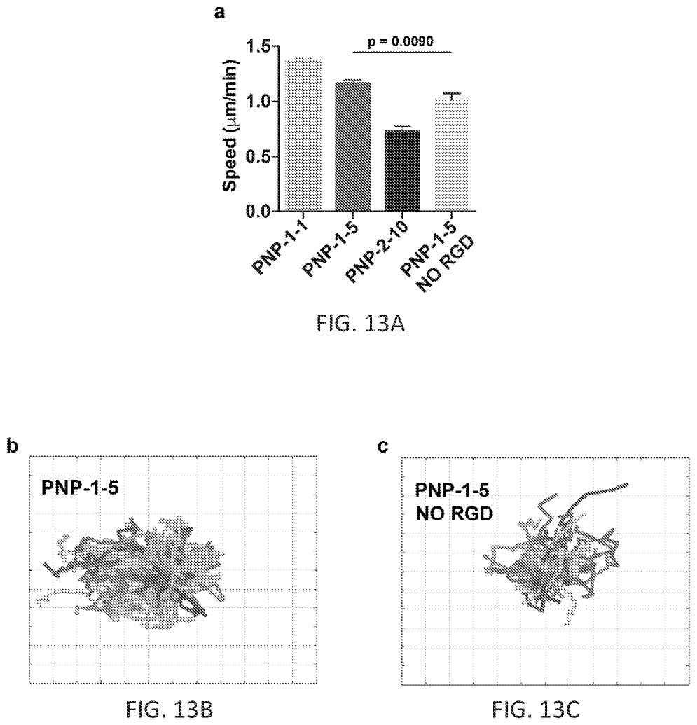

We sought to determine how hydrogel formulation affects the material properties and CAR-T cell motility upon encapsulation (FIG. 1O-FIG. 1P, FIG. 8A-FIG. 10). We prepared three increasingly stiff PNP hydrogel formulations: PNP-1-1, PNP-1-5, PNP-2-10, where the first number denotes the wt % HPMC-C12 and the second number denotes the wt % NPs (remaining wt % is saline). Increasing the concentration of polymer and NPs in the formulation reduces the dynamic mesh size of the matrix, increasing gel stiffness and reducing the matrix self-diffusivity (FIGS. 8A-FIG. 10). As previous studies suggest that migration of T cells encapsulated within a hydrogel environment heavily depends on the mesh size and stiffness of the gels, we conducted cell migration experiments to understand how the dynamic materials properties of the PNP hydrogels impacted the movement of encapsulated CAR-T cells (FIGS. 1L-1P). FIG. 1L shows a testing device 50 with top 56 for testing cell motility. PNP hydrogel with CAR-T cells 14 are placed in testing device 50. Illuminator 52 delivers radiation and movement of cells (indicated by arrows 40, inset) are detected. RGD conjugation to the PNP hydrogel structure improved T cell mobility (FIGS. 13A-13C) and cell migration speed was lower with decreasing hydrogel mesh size and matrix self-diffusivity (FIG. 1O). Based on these studies, the PNP-1-5 formulation was chosen for continuing studies due to its physiologically-relevant stiffness and intermediate cell migration and matrix self-diffusivity characteristics.

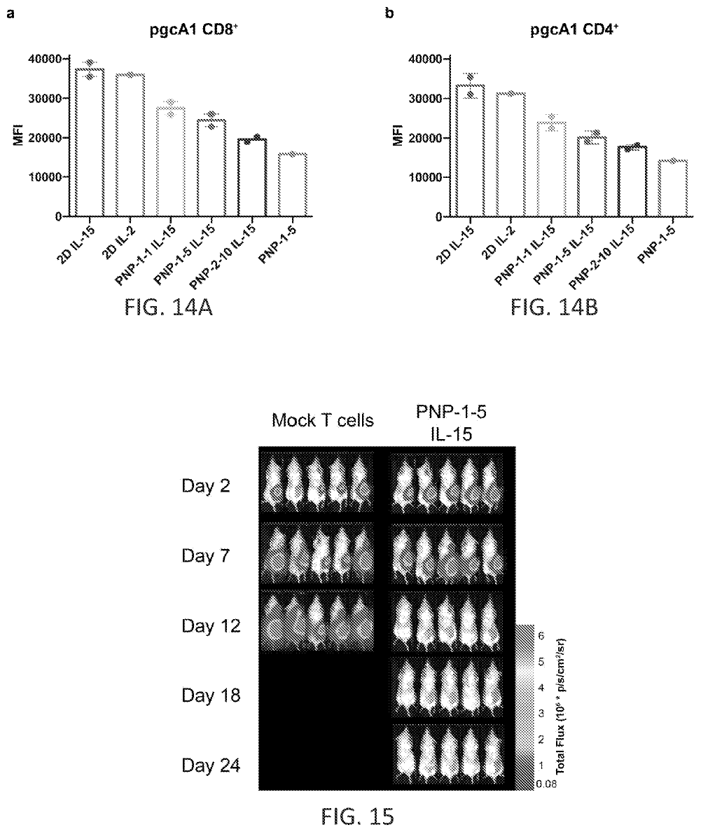

We believed that co-encapsulation of CAR-T cells and stimulatory cytokines such as IL-15, a potent T cell activator that has also been found to support maintenance of T cell memory, would improve CAR-T proliferation within the hydrogels. Moreover, since IL-15 is small (<15 kDa) and unstable, it unfortunately exhibits a short elimination half-life of only 1.5 hours in humans and would benefit from sustained delivery. We first sought to determine whether PNP-1-5 hydrogels could provide extended retention of IL-15 to improve CAR-T activation. In vitro release studies demonstrated that IL-15 was retained within PNP-1-5 hydrogels for over 4 days, exhibiting a release half-life of 2.6 days (FIGS. 11A-11B). We also found that PNP hydrogel encapsulation significantly enhanced the stability of IL-15, corroborating previous findings that PNP hydrogels can improve the stability of encapsulated biologics. Further, in vitro experiments demonstrated that co-encapsulation of CAR-T cells and IL-15 in PNP-1-5 hydrogels enhanced both T cell proliferation within the hydrogels (FIG. 12) and expression of PGC-1α, a master regulator of mitochondrial biogenesis (FIGS. 14A-14B), which could increase their metabolic fitness.

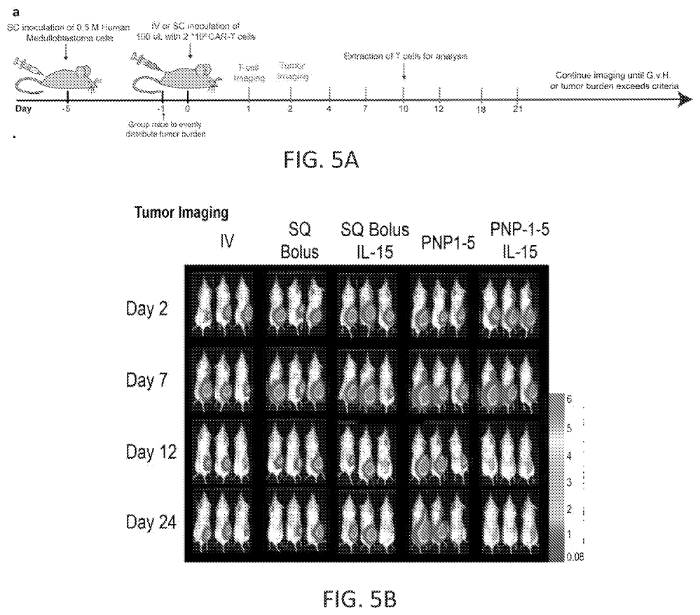

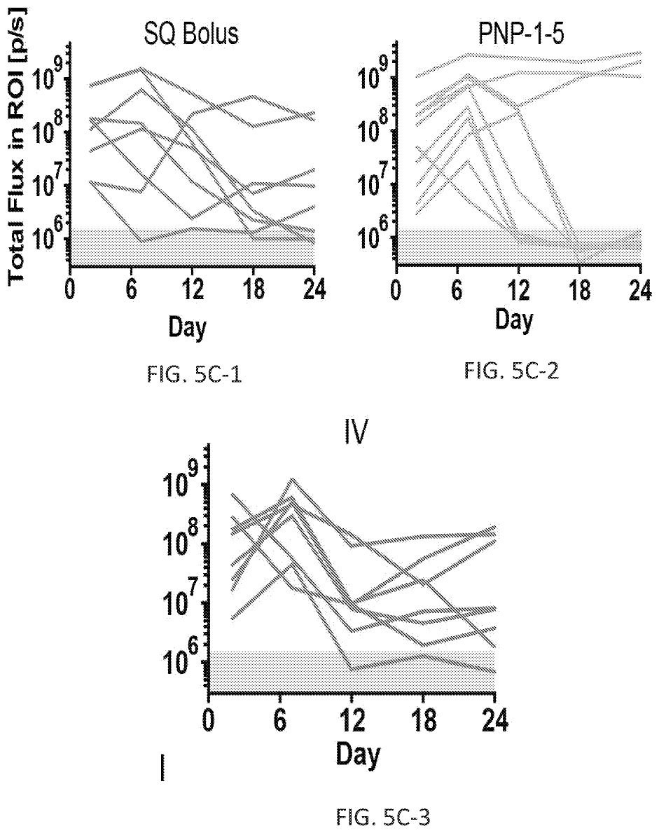

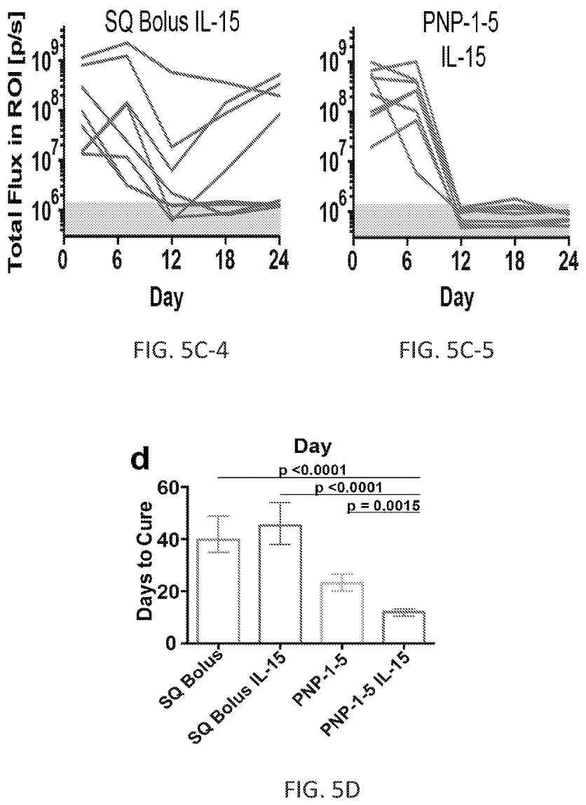

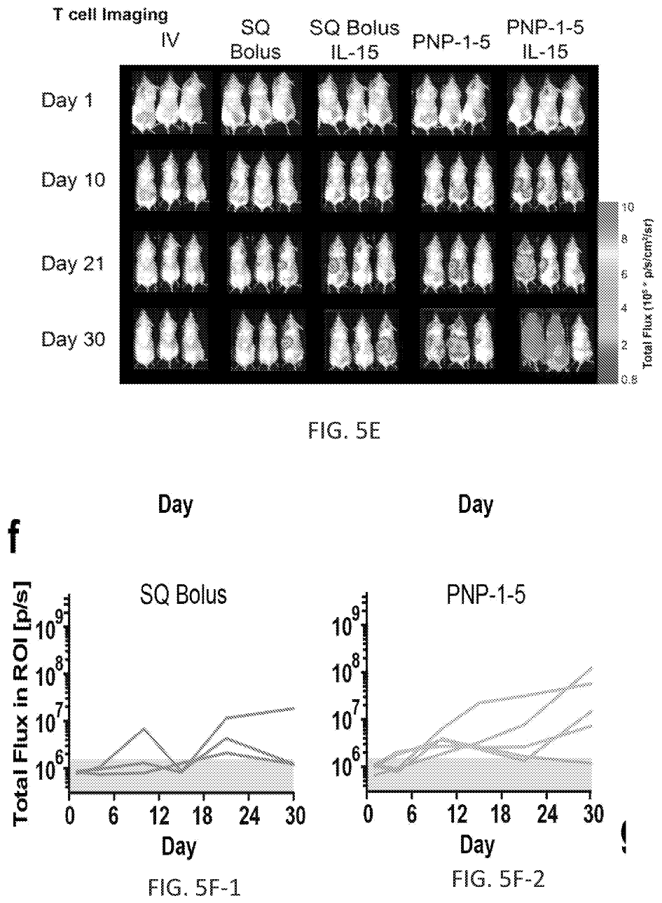

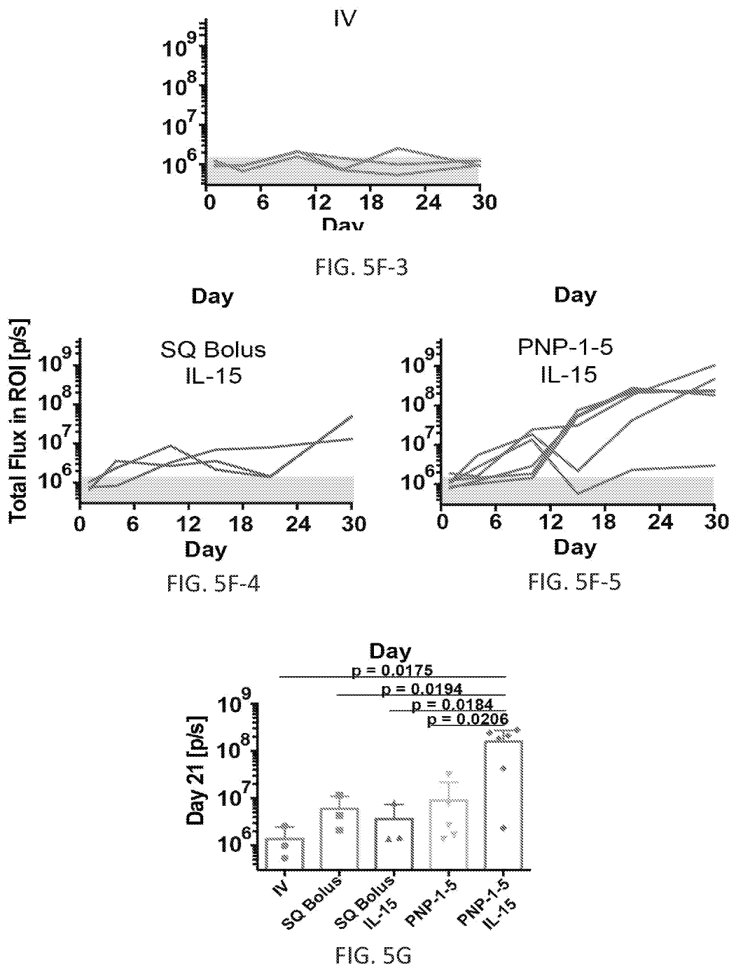

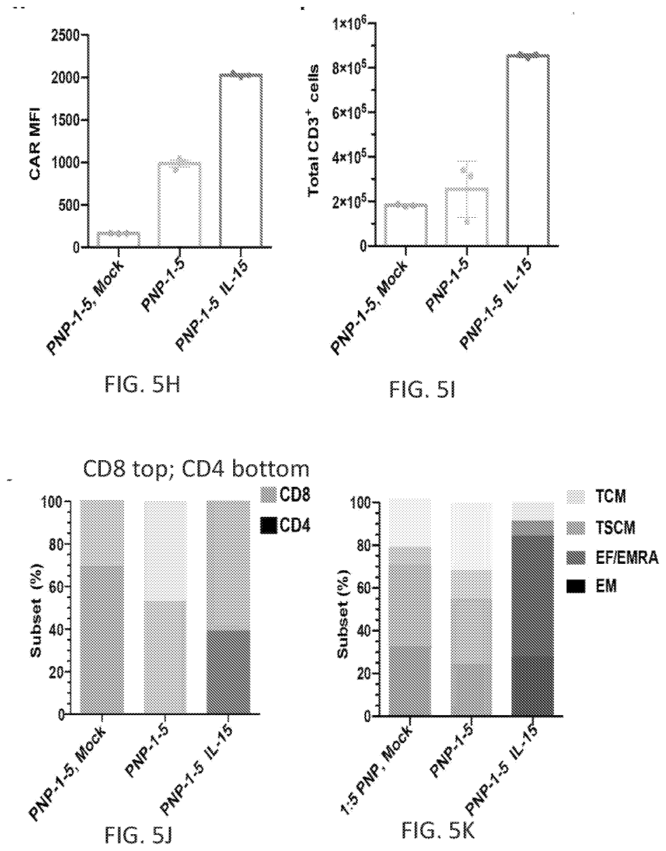

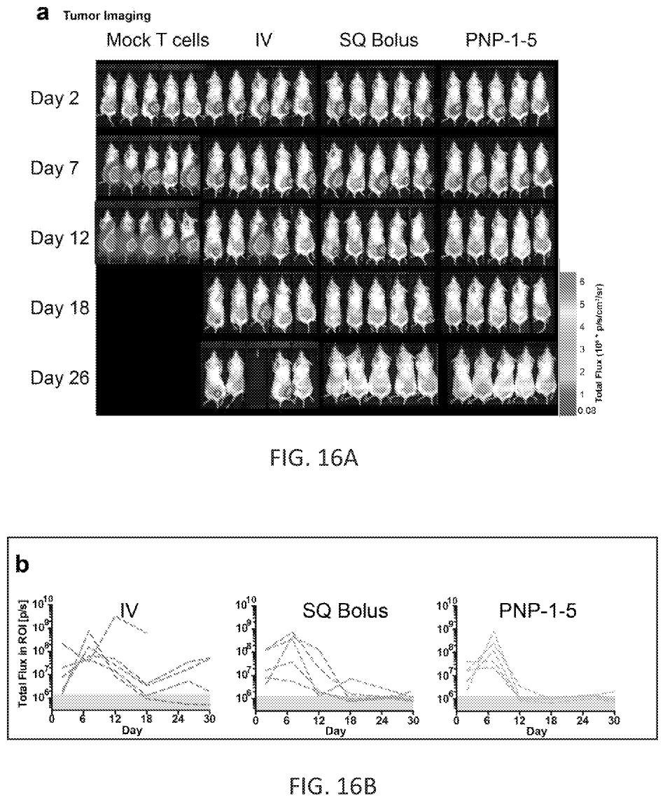

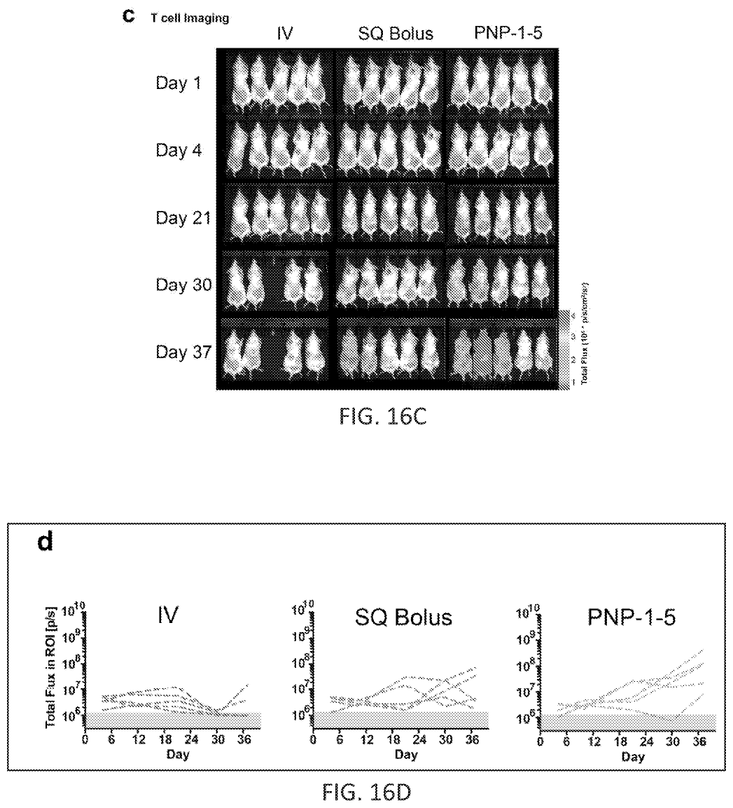

To assess the ability of PNP hydrogels to improve CAR-T cell treatment of solid tumors, human B7H3 CAR-T cells were delivered peritumorally into NSG mice in PNP hydrogels and compared to therapeutically relevant controls in a subcutaneous human medulloblastoma solid tumor model (FIG. 5A). This subcutaneous solid tumor model represents an open, accessible and generalizable location of the body, in contrast to brain or eye tumor models used in recent locoregional delivery studies that are inaccessible. Tumors and CAR-T cells were tracked and quantified in parallel using a dual luciferase in vivo imaging system. PNP hydrogels improved treatment compared to bolus and intravenous therapies (FIGS. 5B-5C, FIG. 15). Bolus and IV controls in a study with more CAR-T cells in each treatment still showed the inability to cure or cured at a slower rate, suggesting a dose-sparing effect where PNP hydrogel groups fewer cells outperformed these controls (FIGS. 16A-16D).

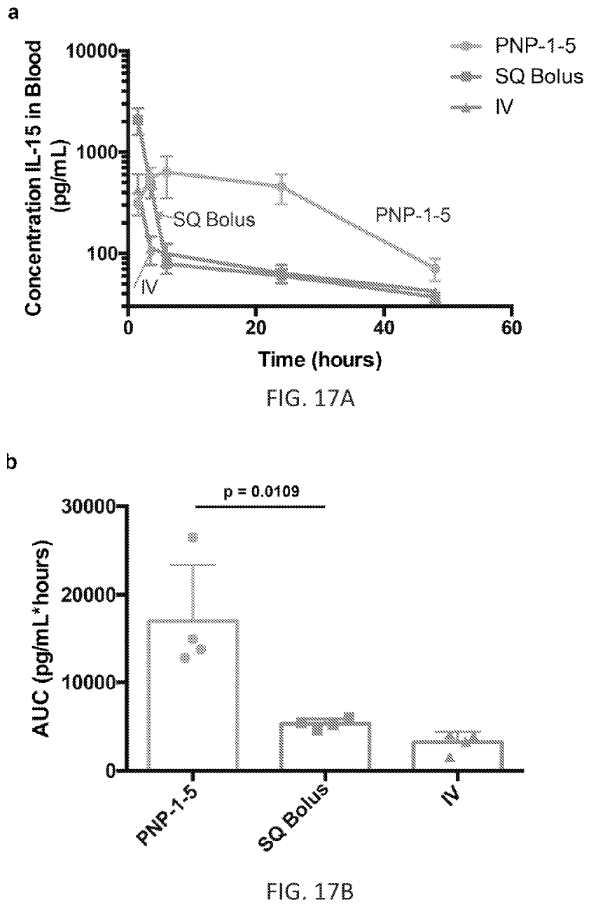

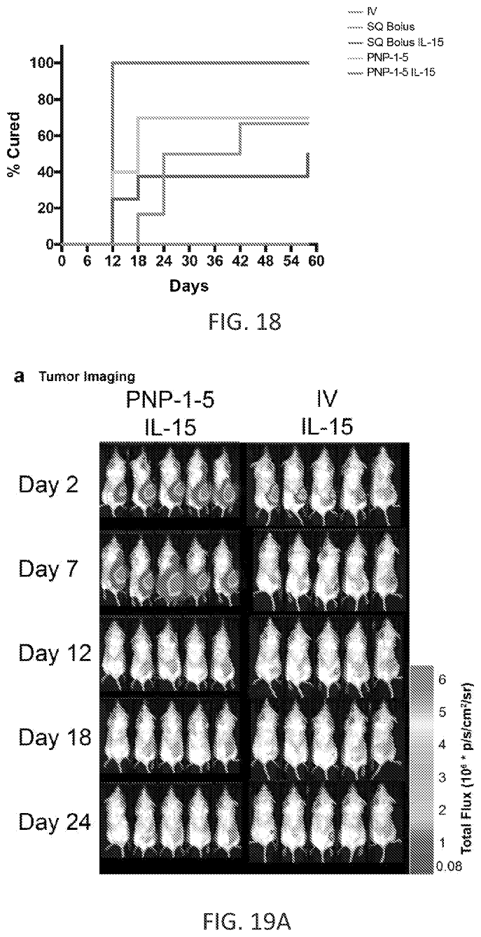

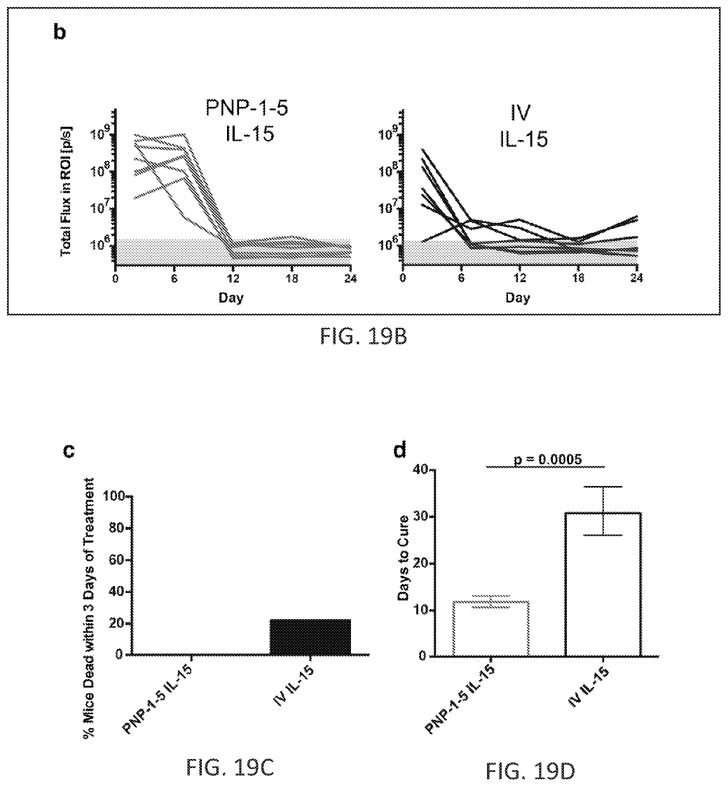

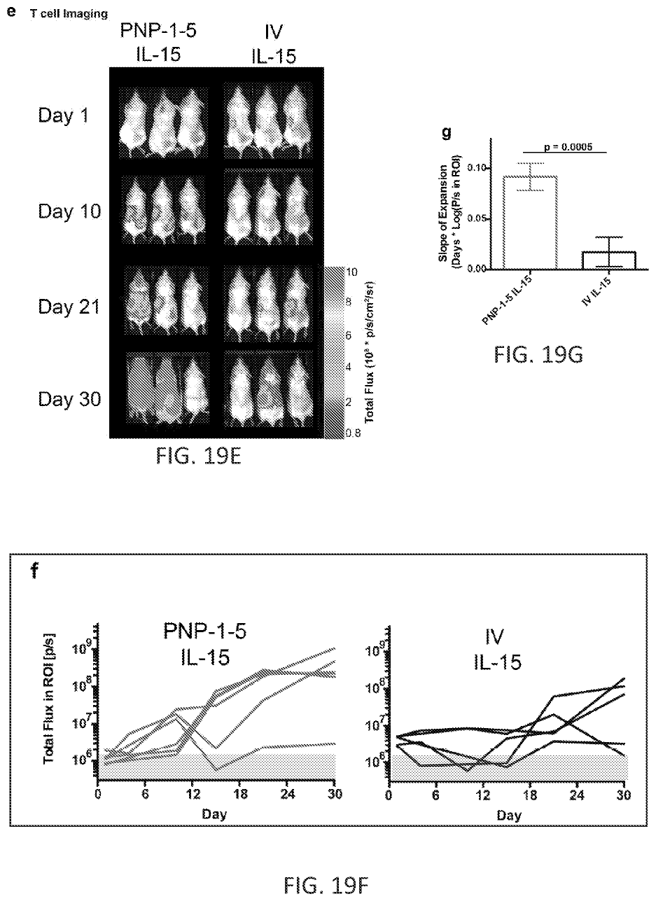

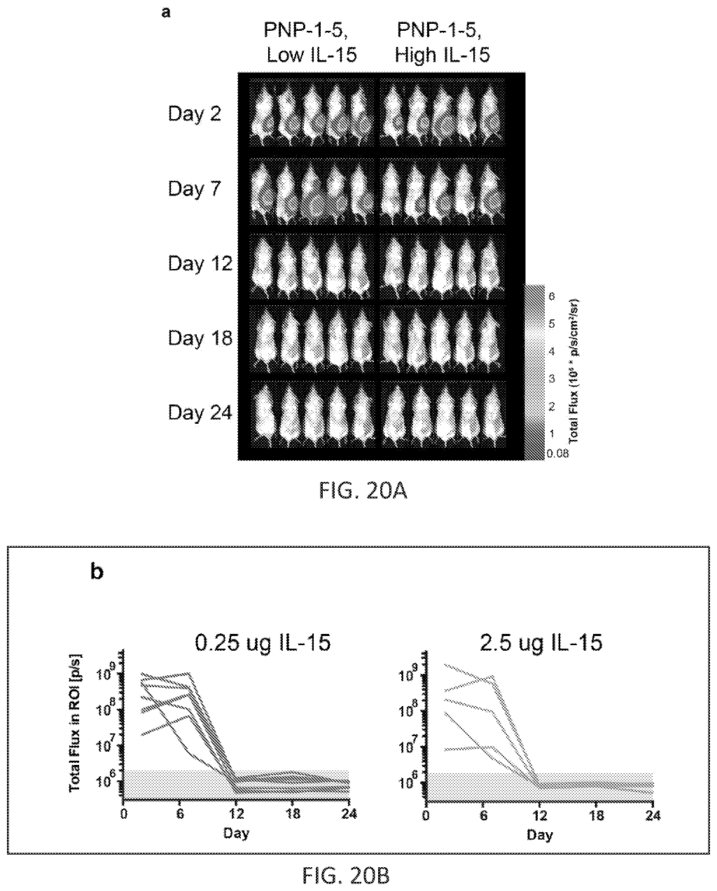

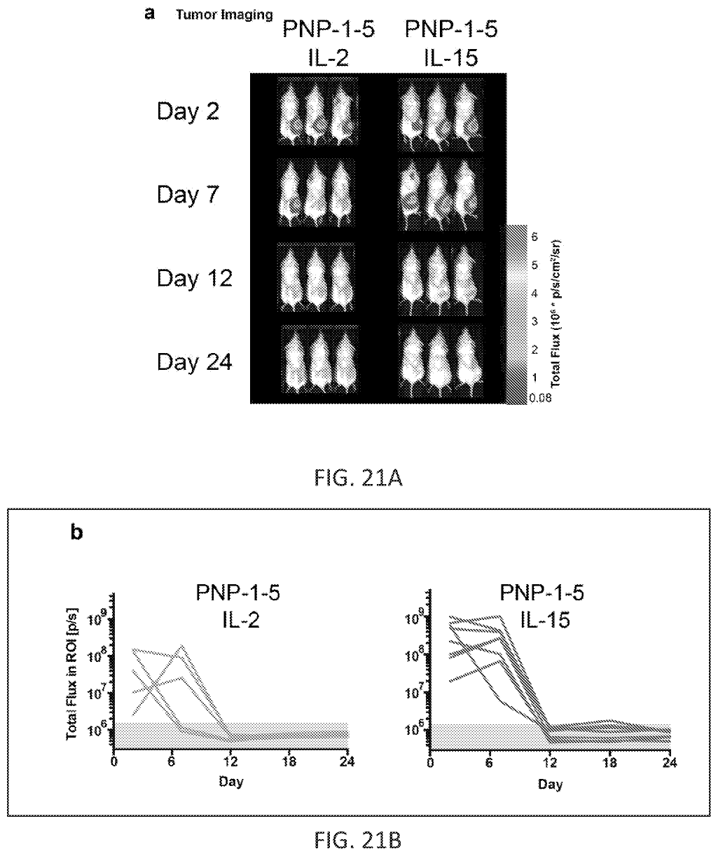

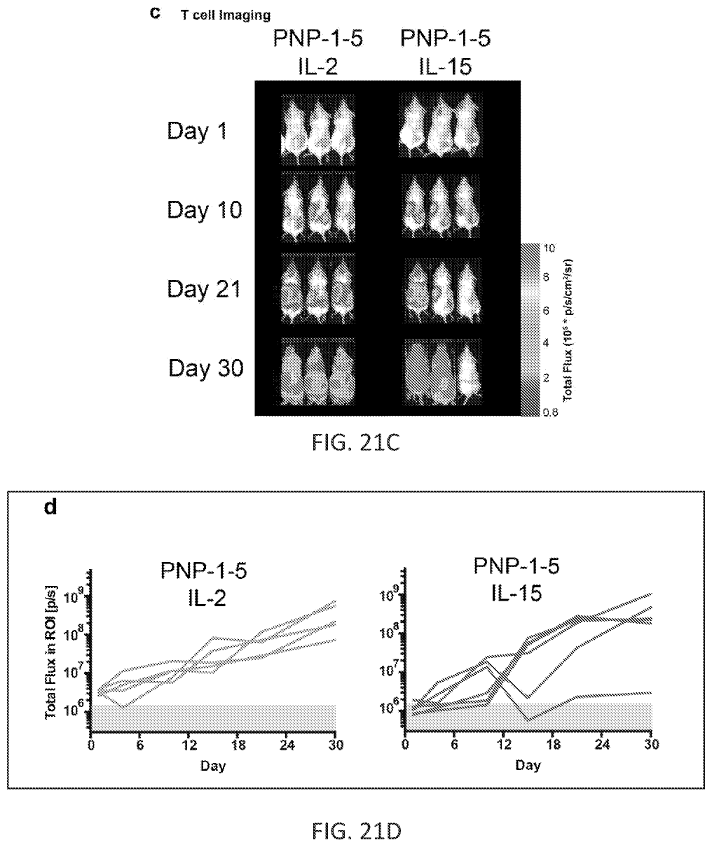

Incorporation of IL-15 in the PNP hydrogel improved treatment (FIGS. 5B-5C). The co-delivered IL-15 dose was below the maximum tolerated subcutaneous dose of rhIL-15 in recent human clinical trials scaled to mice (Supplemental Discussion 1). Systemic IL-15 levels remained elevated in vivo when released from the gel compared to the bolus and intravenous administration (FIGS. 17A-17B). Side effects of the animal model, mainly the emergence of Graft versus Host disease (GVHD) common for ACT of human T cells in NSG mice, limited the length of the study to 30 days. Overall, PNP-1-5 hydrogels with IL-15 improved the cure-rate and consistency of treatment, with all mice completely cured by day 12 (FIGS. 5C-5D, FIG. 18). All other groups exhibited lower efficacy and less consistent results. A treatment including intravenous IL-15 was also investigated. Efficacy was initially comparable to the PNP-1-5 hydrogel with IL-15, but 20% of the mice did not survive the first 3 days after treatment, potentially due to the cytokine concentration spike in the blood. Several mice that did survive also showed tumor relapse (FIGS. 19A-19G). Similarly effective results were observed when co-delivering high concentrations of IL-15 and CAR-T cells in PNP hydrogels (FIGS. 20A-20B) and when co-delivering IL-2, another common activating cytokine, with CAR-T cells in PNP hydrogels as co-delivering IL-15 in PNP hydrogels (FIGS. 21A-21D).

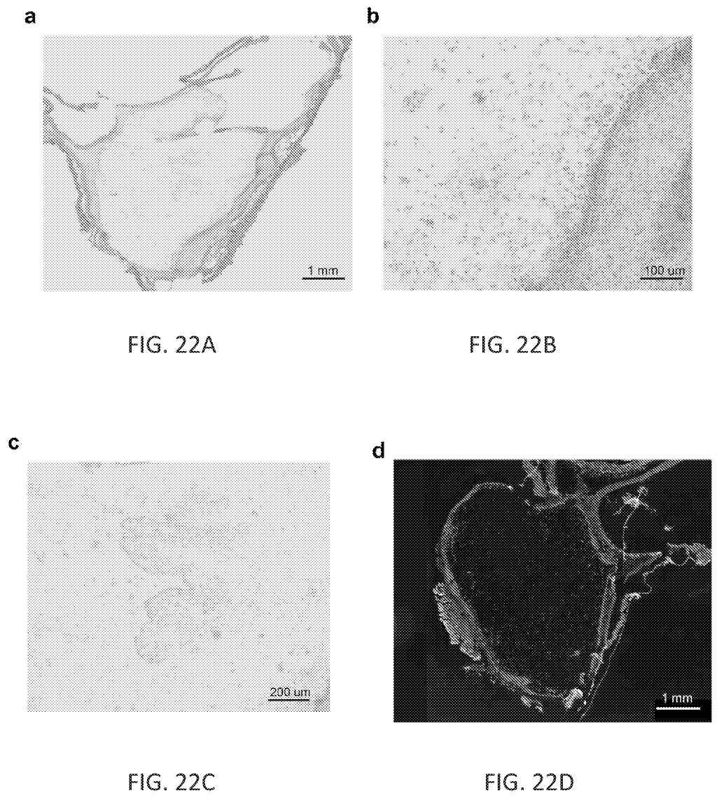

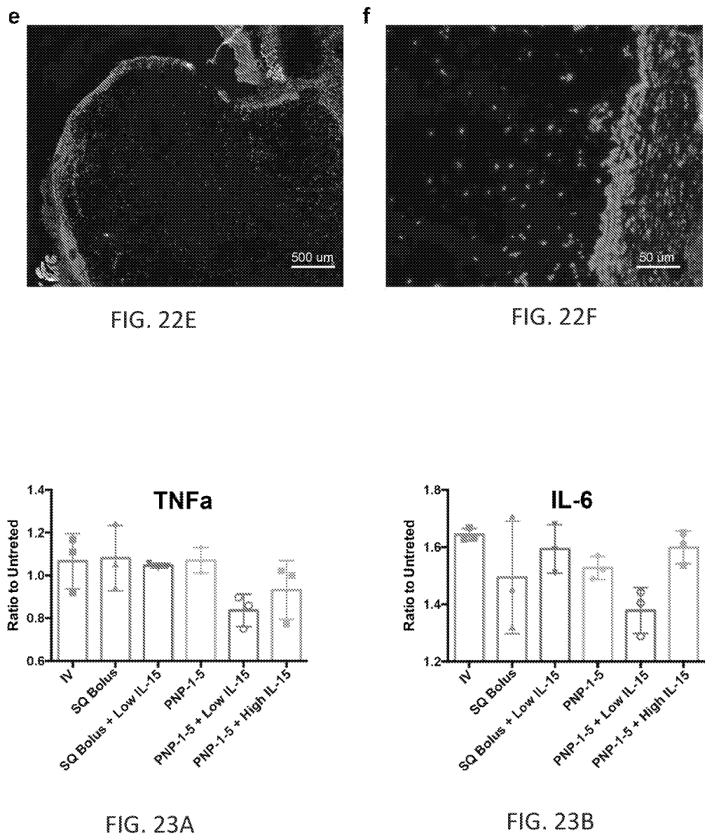

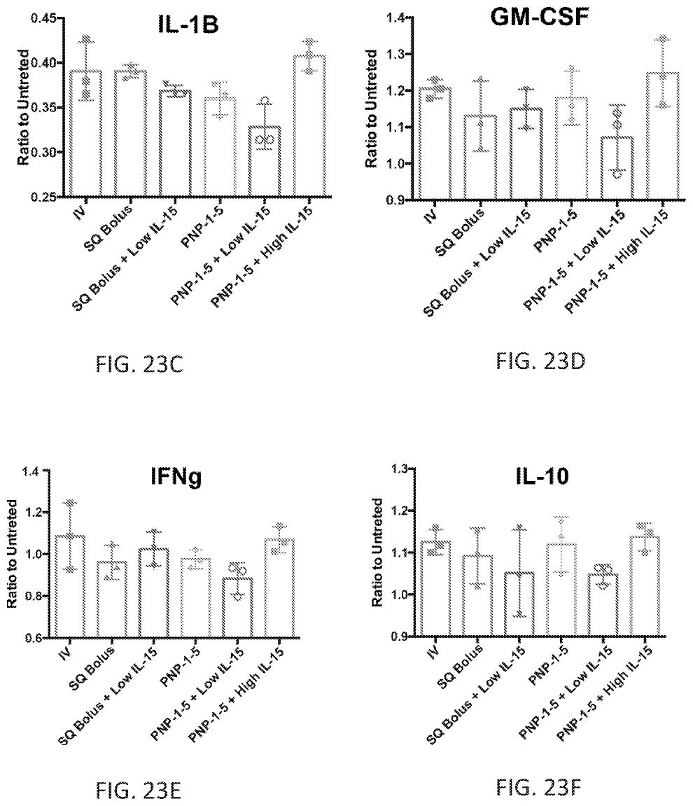

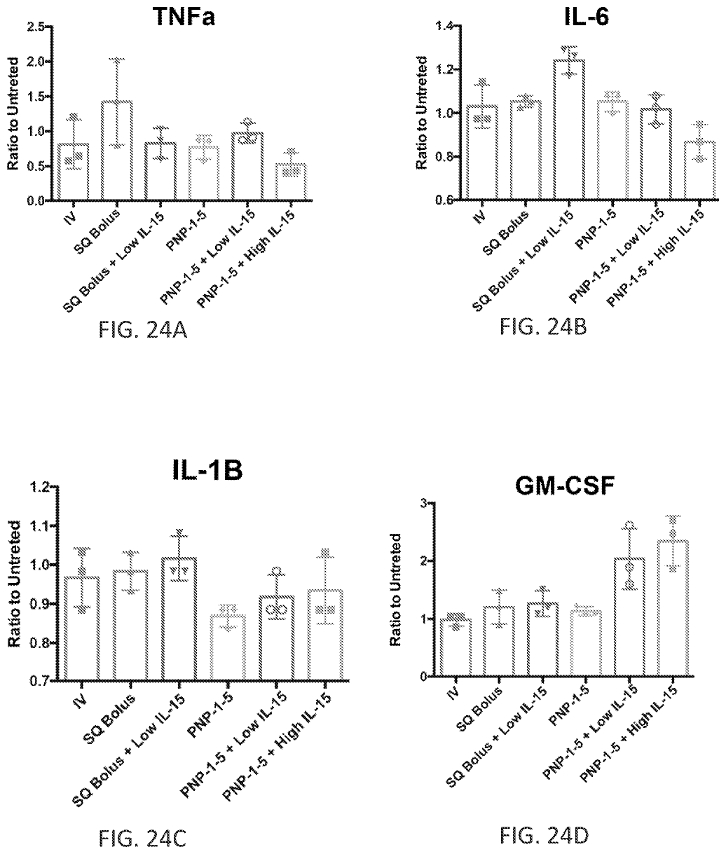

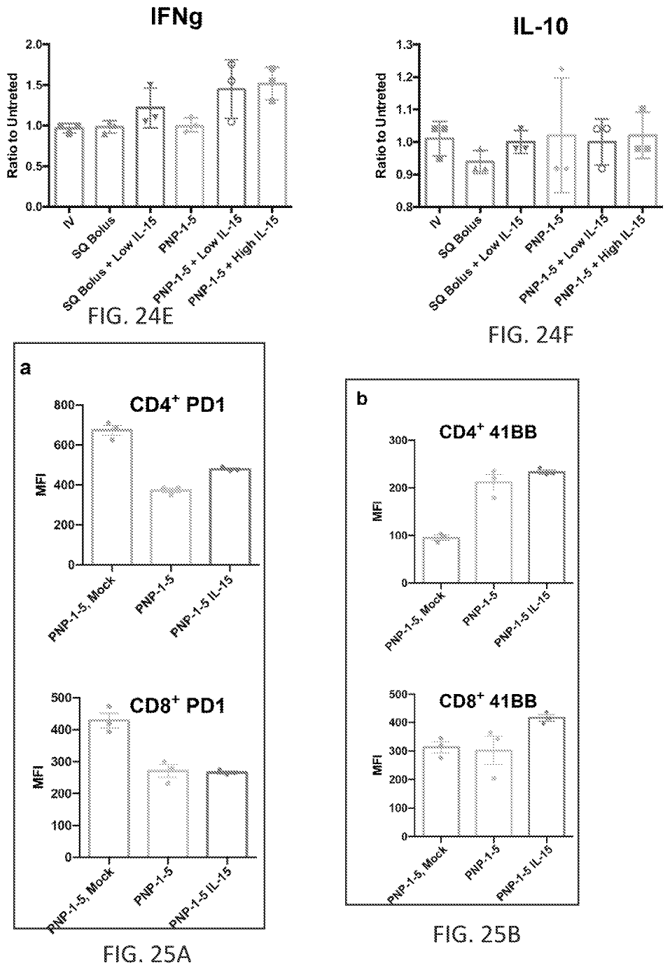

Enhanced T cell expansion was also observed when CAR-T cells were delivered in the PNP hydrogels, particularly when co-delivered with IL-15 (FIGS. 2E-2G). The PNP-1-5 with IL-15 resulted in over a 100-fold increase in T cell expansion compared to the IV control (FIG. 2G). Similar results with increased expansion were observed when delivering higher doses of T cells and when co-delivering IL-2 with CAR-T cells in PNP hydrogels (FIGS. 16A-16D, FIGS. 21A-21D). Gels demonstrated drastically increased T cell expansion at later time points, likely leading to the onset of GVHD. Imaging data show that between days 10 and 21, the primary location of T cell signal moved from the gel on the right subcutaneous flank to the spleen, aligning with the time of tumor eradication. Histology confirmed that there was no adverse immune response to this biomaterial, and that CAR-T cells were still present within the gel and at the periphery of the gel several days after injection (FIGS. 22A-22F). Additionally, inflammatory cytokine analysis confirmed that this treatment method did not elicit spikes in mouse or human cytokines (FIG. 23-FIG. 24).

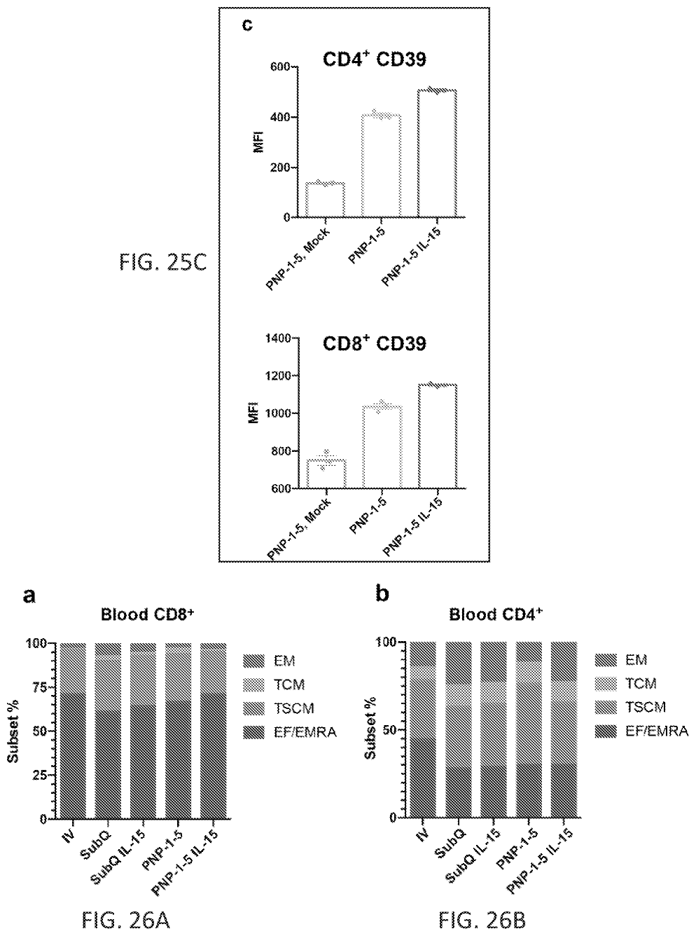

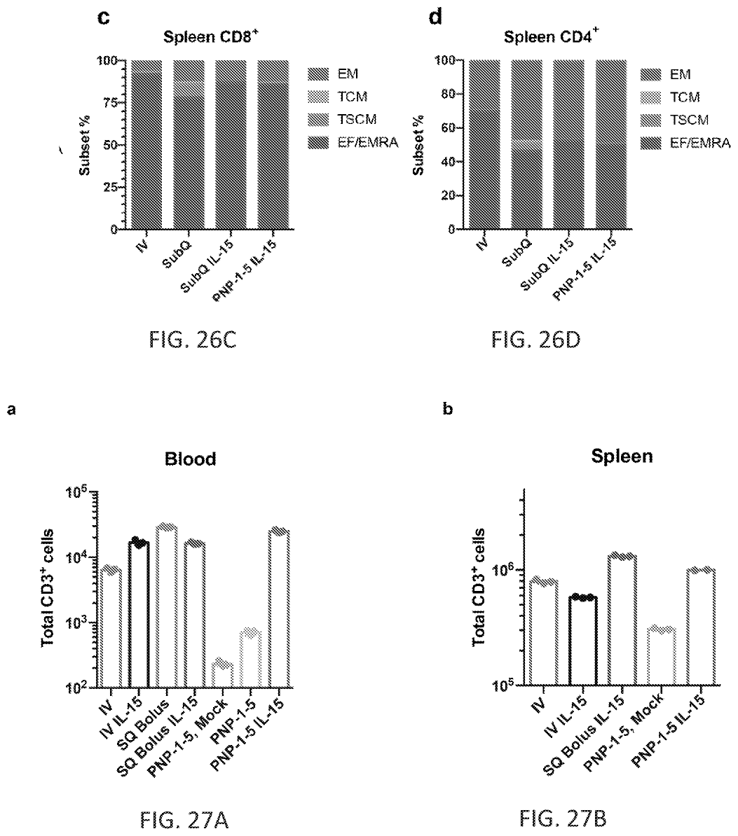

To better understand the mechanism for enhanced efficacy seen in co-delivered CAR-T+IL-15 in PNP-1-5 gels, ex vivo analysis of T cells was performed 10 days after treatment. This analysis revealed enhanced CAR expression (FIG. 5H), increased persistence (FIG. 5I), and greater proportions of CD8+(FIG. 5J) and TSCM subsets (FIG. 5K) from T cells co-encapsulated with IL-15. While all CAR-T groups had similar levels of the activation markers 4-1BB, LAG-3, and PD-1, co-delivery of IL-15 resulted in enhanced expression of CD39, which has recently emerged as a marker of tumor-reactive T cells (FIGS. 25A-25C). Co-delivered IL-15 increased T cell numbers in blood and spleen samples of PNP CAR-T treated mice, whereas memory subsets were similar between all groups at these sites (FIGS. 26A-26D and FIGS. 27A-27B).

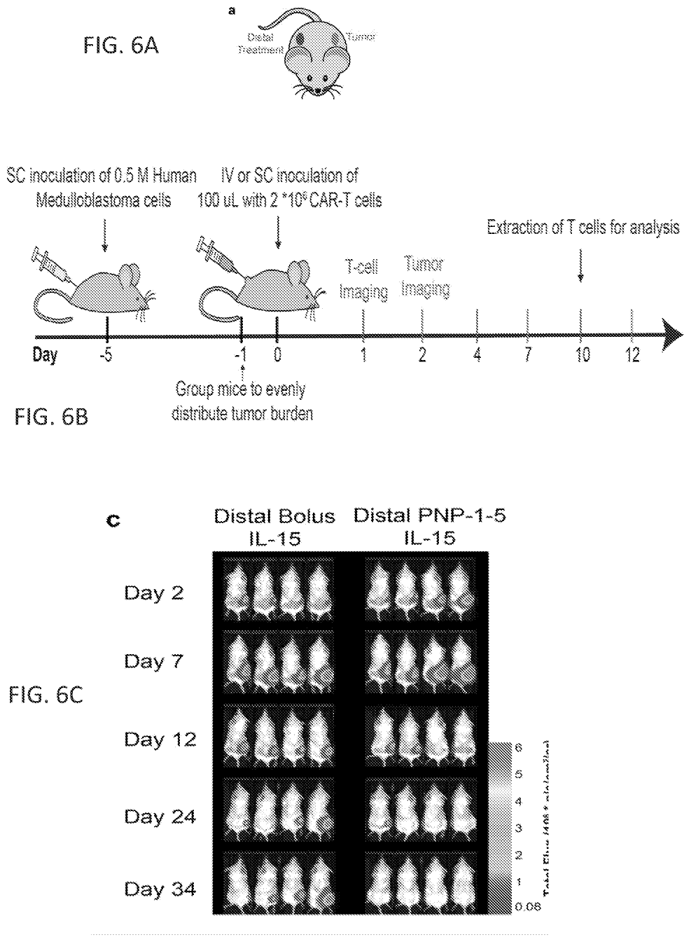

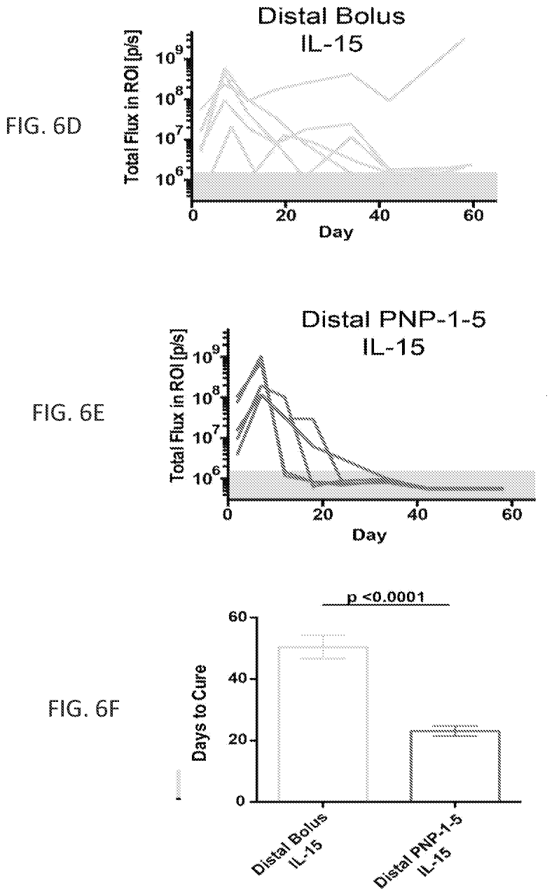

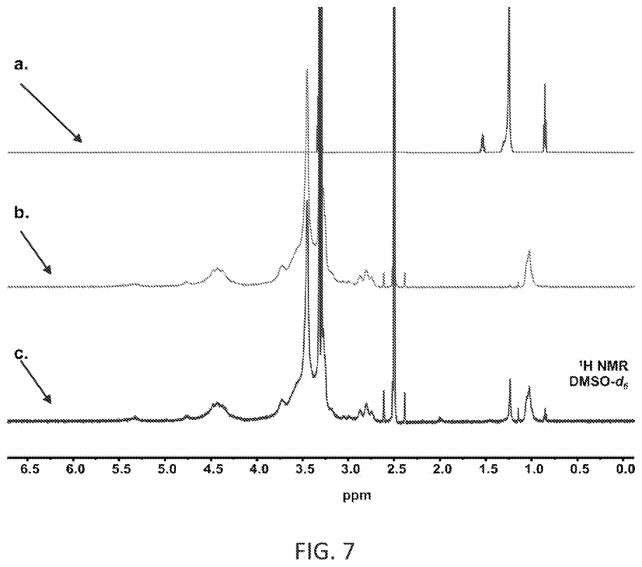

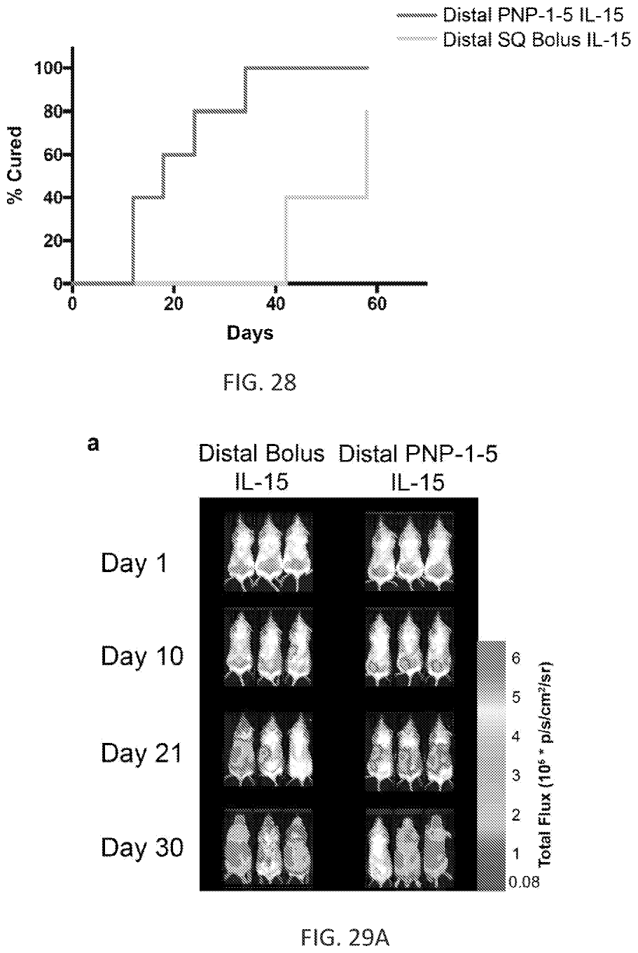

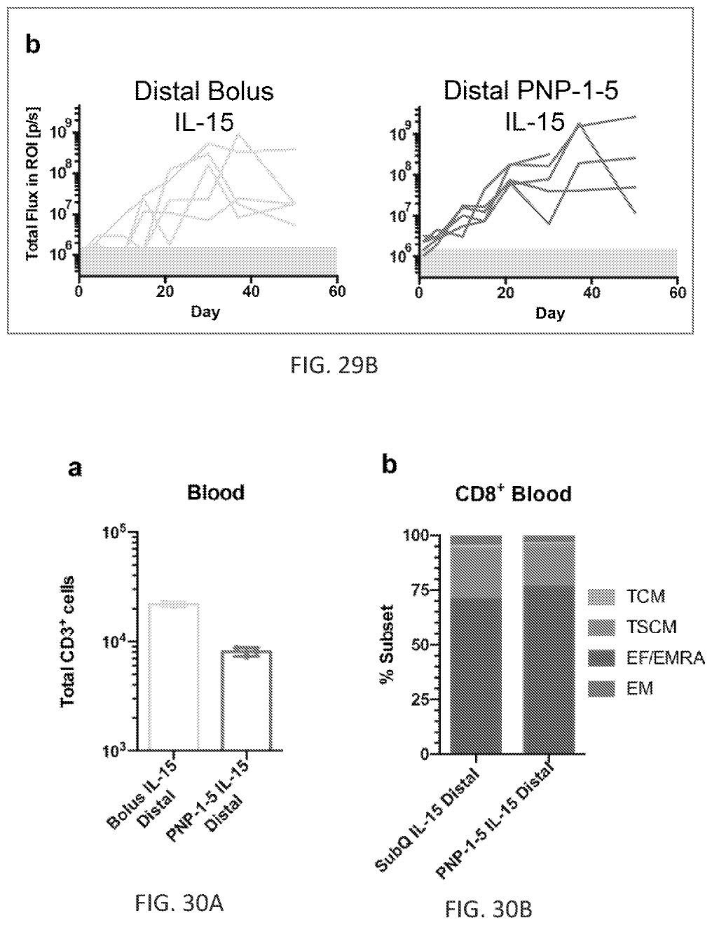

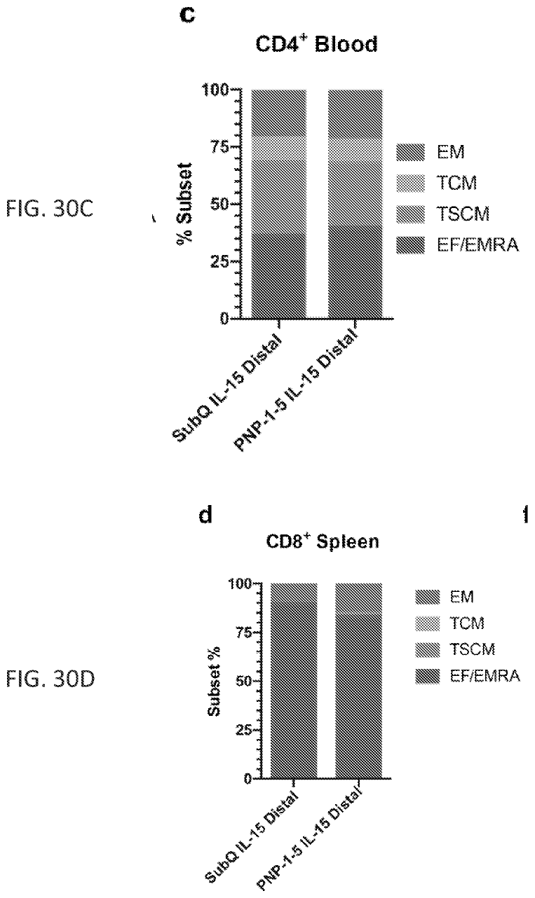

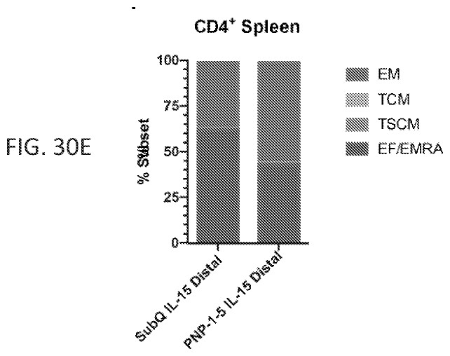

In addition to delivering the hydrogel peritumorally, experiments were also performed in which the hydrogel was delivered subcutaneously on the distal flank of the mouse so that the T cells released from the gels would not drain directly to the tumor (FIGS. 6A-6F and FIG. 28). Imaging studies revealed that the hydrogel containing IL-15 led to improved tumor clearance over the distal bolus control even though the two treatments exhibited similar overall T cell expansion and a similar distribution of T cell subsets in blood and spleens (FIGS. 29A-29B and FIG. 30). These studies had a longer duration than previous studies as mice experienced significantly delayed GVHD. Further, while the distal hydrogel treatment displayed delayed efficacy compared to the peritumoral hydrogel treatment, all mice were ultimately cured. These promising results suggest that this hydrogel treatment could have future use in treating metastatic cancers or inaccessible tumors.

Unless otherwise indicated directly or by context, the materials and methods described below were used for performing the experiments described herein.

Materials: All chemicals, reagents, and solvents were purchased as reagent grade from Sigma-Aldrich, Acros, or Alfa Aesar and used as received unless otherwise specified. Glassware and stir bars were oven-dried at 180° C. When specified, solvents were degassed by three cycles of freeze, pump, and thaw. HPMC-C12, PEG-PLA, and RGD-PEG-PLA were synthesized and characterized as described previously. NPs were prepared by nanoprecipitation according to literature procedures using a 50:50 mixture of RGD-PEG-PLA:PEG-PLA poylmers, and NP size and dispersity were characterized by dynamic light scattering (diameter=35 nm, PDI=0.02).

Hydrogel formulation and cell Encapsulation: Procedure was followed and analyzed as described previously. HPMC-C12 was dissolved in phosphate-buffered saline at 6 wt % and loaded into a 1 mL luer-lock syringe. A cell pellet containing the number of cells to reach the desired concentration in the final hydrogels was suspended in phosphate-buffered saline. A 20 wt % nanoparticle solution in PBS was then added to the cell suspension. A 20:80 mixture of RGD to plain NPs were used to form the hydrogel for studies containing RGD, yielding a 0.5 mM concentration of conjugated RGD in the gel. The CAR-T cell/nanoparticle solution was loaded into a 1 mL luer-lock syringe. The cell/nanoparticle syringe was then connected to a female-female mixing elbow and the solution was moved into the elbow until it was visible through the other end of the elbow. The syringe containing the HPMC-C12 polymer was then attached to the elbow other end of the elbow. The two solutions were then mixed gently back and forth through the elbow for 30 seconds to 1 minute until the solutions had completely mixed and formed a homogeneous cell-loaded PNP hydrogel. IL-15 (R&D Systems) was incorporated with the nanoparticles during hydrogel formulation.

Rheological characterization of hydrogels: Rheological testing was performed using a 20 mm diameter serrated parallel plate at a 600 μm gap on a stress-controlled TA Instruments DHR-2 rheometer. All experiments were performed at 25° C. Frequency sweeps were performed at a strain of 1%. Amplitude sweeps were performed at frequency of 10 rad/s. Flow sweeps were performed from high to low shear rates with steady state sensing with 5 points within 10% within 120 seconds.

Cell lines: MED8A was kindly provided by S. Chesier (Stanford University, Stanford, CA). MED8A-GFP-Fluc cells were cultured in DMEM supplemented with 20% FBS, 100 U/mL penicillin, 100 μg/mL streptomycin, 2 mM L-glutamine, and 10 mM HEPES (Gibco). STR DNA profiling was conducted once per year (Genetica Cell Line testing) and routinely tested for Mycoplasma. Cell lines were cultured in a 5% CO2 environment at 37° C.

Plasmid construction and virus production: B7H3 CAR-P2A-Nluc plasmid was constructed by fusing the MGA271 scFv to CD8a hinge and transmembrane, 4-1BB costimulation domain, CD3ζ signaling domain, porcine teschovirus-1 2A (P2A) ribosomal skipping sequence, and nanoluc in an MSGV retroviral vector. The Antares-P2A-mNG constructed was constructed by fusing a P2A sequence and mNeonGreen to the c-terminus of Antares. Retroviral supernatant was produced using 293GP packaging cells transfected with the RD114 envelope plasmid and the corresponding plasmid construct, as previously described.

CAR-T cell isolation: T cells were isolated from buffy coats purchased from the Stanford Blood Center under an IRB-exempt-protocol. Negative selection using the RosetteSep Human T cell Enrichment kit (Stem Cell Technologies) and SepMate-50 tubes was performed to purify primary human T cells. T cells were crysperserved in CryoStor CS10 media at a concentration of 1-2×107 cells/mL.

CAR-T manufacturing: Primary human T cells were thawed at Day 0 and activated with anti-CD3/CD28 Dynabeads (Thermo Fisher) at a 3:1 bead to T cell ratio and cultured in AIM V+5% heat-inactivated FBS, 100 U/mL penicillin, 100 mg/mL streptomycin, 2 mM L-glutamine, 10 mM HEPES, and 100 U/mL rhIL-2). On Day 2, virus-coated wells were prepared on 12-well non-tc, Retronectin-coated (Takara/Clontech) plates by spinning 1 mL of the corresponding virus at 3200 RPM for 2 hours. T cells were then cultured on these plates for 24 hours. This transduction process was on Day 3 after. Beads were magnetically removed on day 4, and cells were expanded until Day 10 for in vivo experiments and Day 10-14 for in vitro experiments. For dual virus cotransductions, T cells were transduced with Antares-P2A-mNG on Day 2 and B7H3 CAR-P2A-Nluc on Day 3.

Ex vivo CAR-T cell analysis: For ex vivo analysis of transferred T cells, mice were euthanized ten days after T cell administration. Gels were harvested and T cells were extracted by mechanical dissociation (gentleMACS dissociator, Miltenyi). Single-cell suspensions were filtered and stained for flow cytometry.

Flow cytometry: B7H3 CAR was detected using recombinant B7H3-Fc (RD Systems) fluorescently labeled with the DyLight 650 Microscale Antibody Labeling Kit (Thermo Fisher). The following antibodies were used to stain T cells: BUV395 Mouse Anti-Human CD4 (Clone SK3, BD), BUV805 Mouse Anti-Human CD8 (Clone SKi, BD), BV605 Mouse Anti-Human CD62L (Clone DREG-56, BD), and BV711 Mouse Anti-Human CD45RA (Clone HI100, BD). CAR-T cell quantification was performed using the CountBright Absolute Counting Beads (Thermo Fisher). Flow cytometry was performed on a BD Fortessa and analyzed on FlowJo version 10.7.1.

WST proliferation assay: 100 μL of PNP-1-5 hydrogel was loaded in wells of a 96 well plate with varying concentrations of IL-15 (0, 0.25 μg/mL and 2.5 μg/mL) at 0.5 million cells/mL. 100 μL of CAR-T cell media was placed on top of each gel and cultured at 37° C. and 5% CO2. After one day, the media was removed and 100 μL of new media and 10 μL of WST reagent was added to each well (WST-1 Cell Proliferation Reagent, ab155902). After 2 hours of incubation in the WST solution, the absorbance was read using a plate reader at OD=450 nm after shaking for 10 seconds.

CAR-T cell migration studies: Live cell imaging was done with a laser scanning confocal microscope (Leica SP8) fitted with temperature/incubator control, suitable for live-imaging (37° C., 5% CO2). A 20×-air objective, NA=0.75, was used to acquire approximately 60 μm stack images for 5 hours (at 10 minute intervals). Imaging parameters were adjusted to minimize photobleaching and avoid cell death. After completion of migration studies, the centroids of mCherry-labeled cells were tracked using the spots detection functionality in Imaris (Bitplane). Poorly segmented cells and cell debris were excluded from the analysis and drift correction was implemented where appropriate. A custom MATLAB script was used to reconstruct cell migration trajectory.

Fluorescence recovery after photobleaching: Three different PNP hydrogel formulations were prepared with distinct HPMC-C12 and NP concentrations (PNP-2-10, PNP-1-5, and PNP-1-1). Fluorescein isothiocyanate was coupled to HPMC-C12 according to literature protocols 21. Gels were placed onto glass slides and imaged using a confocal LSM780 microscope. Samples were imaged using a low intensity laser to observe an initial level of fluorescence. Then the laser was switched to full intensity and focused on a region of interest (ROI) with a 25 μm diameter for 10 seconds in order to bleach a circular area. Fluorescence data were then recorded for 4 minutes to create an exponential fluorescence recovery curve. Samples were taken from different regions of each gel (n=5-9). The diffusion coefficient was calculated according to the following equation:

$${D}^{= \frac{{γ}_{D} {ω}^{2}}{4 {τ}_{1 / 2}}}$$

τ1/2 where the constant D=τD with τ_{1/2} being the half-time of the recovery, τD the characteristic diffusion time, both yielded by the ZEN software, and ω the radius of the bleached ROI (12.5 μm).

In vivo experimental approaches: All experiments followed approved protocols. 5×105 MED8A-GFP-Fluc cells were injected s.c. on the right flank of NSG mice 5 days prior to treatment in a 50:50 ratio of Matrigel (Cultrex Pathclear) to phosphate buffered saline (PBS). One day prior to treatment mice were imaged and distributed into groups of roughly equivalent tumor burden. CAR-T cells were encapsulated in phosphate buffered saline or PNP hydrogels in 1 mL syringes in a tissue culture hood one hour before injection as previously described. One syringe was prepared for each experimental group within a study. 300 μL extra gel was prepared in each syringe. Double the volume of bolus and intravenous controls was prepared. Syringes were transported on ice to the animal facility. 100 μL of phosphate buffered saline or PNP hydrogel was delivered to each mouse. Subcutaneous injections were delivered in a 21 G luer lock syringe.

In vivo imaging: To image tumors, mice were intraperitoneally injected with D-Luciferin, potassium salt (Goldbio) at 150 mg/kg in phosphate buffered saline. After 5 minutes, mice were anesthetized with isoflurane gas and imaged with an exposure time of 30 seconds with an In Vivo Imaging System (Spectral Imaging Instruments Lago-X). Signal was quantified as the total flux of photons/sec in the region of interest at peak intensity. The region of interest was defined as a rectangular box of consistent size around the entire mouse. Background signal for quantification was defined as the maximum signal observed through all imaging experiments in an equivalently sized rectangular box with no luminescent signal. To image tumors, mice were intraperitoneally injected with nano-luciferin (NanoLuc, Promega) at a 40× dilution in phosphate buffered saline. After 5 minutes, mice were anesthetized with isoflurane gas and imaged with an exposure time of 30 seconds In Vivo Imaging System (Spectral Imaging Instruments Lago-X). Signal was quantified as the total flux of photons/sec in the region of interest at peak intensity. The region of interest was defined as a rectangular box of consistent size around the entire mouse.

Histology: Gels were explanted through dissection from mice on Day 5 of treatment and frozen in optimal cutting temperature compound (OCT). All samples were processed and stained by Stanford Animal Histology Services. Two replicates were collected from PNP-1-5 hydrogel containing IL-15, and two replicates were collected for PNP-1-5 hydrogel.

Cytokine release in vitro: Capillary tubes were loaded with 100 μL of PNP hydrogel containing 0.25 μg IL-15. 300 μL phosphate buffered saline (PBS) was loaded on top of each gel. Samples were stored at 37° C. to mimic physiological environments. At each time point, the PBS was completely removed using a long needle and stored at −80° C. for later analysis. The PBS was then replaced. IL-15 concentrations were determined by ELISA according to the manufacturer's instructions (R&D Systems Human IL-15 Quantikine Assay). Absorbance was measured at 450 nm in a Synergy H1 Microplate Reader (BioTek). At the end of 4 days, the gel was diluted and analyzed for remaining cytokine. Cytokine concentrations were calculated from the standard curves. Mass in gel was calculated as the inverse of the total mass released into the release buffer during the study and the cytokine left in the gel at the end of the study. Half-life was calculated by fitting to an exponential decay.

Cytokine release in vivo: Serum was collected at the indicated times by tail vein blood collection and stored at −80° C. Serum IL-15 concentrations were determined by ELISA according to the manufacturer's instructions (R&D Systems Human IL-15 Quantikine Assay). Absorbance was measured at 450 nm in a Synergy H1 Microplate Reader (BioTek). Cytokine concentrations were calculated from the standard curves.

Inflammatory cytokine Analysis: 60 μL blood was collected after 48 hours from each mouse (n=3 for each group). Samples were processed and analyzed by the Stanford Human Immune Monitoring Center. Samples are reported relative to naive mice.

Cure-rate analysis: To test if time to cure differed between treatments, we used a maximum likelihood parametric regression (PROC LIFEREG) with censored data in SAS University Edition. Mice were counted as cured if their signal reached and stayed below 1.5×106 p/s. Mice who were euthanized due to graft versus host disease, tumor size, or early study termination as a result of COVID-19 shutdowns were right-censored. No mice in the IV treatment group were cured before the experiment end and were all censored, thus IV treatment was excluded from this analysis. Since some groups were evaluated over multiple experimental runs, experiment cohort was included in the model as a fixed blocking (control) factor. Initial tumor size was also included in the model as a blocking factor. Least-squared means were used to compare time to cure between individual treatments and Tukey-Kramer post-hoc tests were used to correct for multiple comparisons.

CAR-T cell expansion analysis: For statistical analysis, the T cell luminescence readings required additional log 10 transformation to meet the assumptions of homoscedasticity. To test if CAR-T cell population growth differed between treatments, we used a restricted maximum likelihood (REML) mixed model in SAS University Edition. Mouse was included as a random effect subject and experiment cohort was included as a fixed blocking (control) factor. The interaction between treatment and time tested whether treatment altered tumor growth over time. Bonferroni correction was used to adjust for multiple comparisons (α=0.0033).

Statistical methods: All error is reported as standard deviation unless reported otherwise.

Calculation of rhIL-15 Dose Equivalence in Humans: While many CAR-T therapies require lymphodepletion, there has been little research on how lymphodepletion affects the maximum tolerated dose of rhIL-15, so the dosage in our studies is based on the available literature. The maximum tolerated subcutaneous dose per day in recent human clinical trials is 2 μg/kg/day. We can scale this dose to mice by multiplying by 12.3, giving 24.6 μg/kg/day. Assuming mice are approximately 0.02 kg in weight, gives 0.492 g in one dose in a mouse. We chose to administer approximately half this dose, 0.25 μg, subcutaneously in our studies. Note that this dose exceeds the maximum tolerated dose of rhIL-15 intravenously delivered in humans, 0.3 μg/kg/day, 2 equating to 0.073 μg.

While CAR-T therapy has resulted in remarkable anti-tumor effects in B cell malignancies, clinical activity in solid tumors has been limited. This is partly due to an unfavorable solid tumor microenvironment, which can contain factors that inhibit T cell activation and persistence and promote dysfunction. Our novel gel-based delivery method provides a local niche that helps to expand and maintain a reservoir of transferred T cells and to improve efficacy. Future studies are required to investigate formulations comprising additional factors such as immunostimulants and checkpoint inhibitors that could help to recruit endogenous immune cells to further amplify the response and promote epitope spreading.

The co-delivery of CAR-T cells and cytokines in PNP hydrogels as describe herein provides a strategy for treating solid tumors. This scalable and injectable material provides minimally invasive delivery of CAR-T cells to improve treatment of solid tumors and reduce the number of cells required for effective treatment and in-turn the cost associated with extended manufacturing periods. In vitro studies elucidated the design criteria for hydrogel formulation. PNP hydrogels can simultaneously slowly release CAR-T cells and cytokines, enhance stability of cytokines, and improve local T cell expansion, leading to improved efficacy. PNP hydrogels improved CAR-T cell treatment both local and distal to the tumor. PNP hydrogels address and unmet need for effective CAR-T cell delivery to treat local and distal solid tumors.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An immunotherapy delivery system, comprising:
a hydrogel comprising a polymer non-covalently crossed-linked with a plurality of nanoparticles;
a first immunomodulatory cargo comprising cells encapsulated in the hydrogel;
a cell adhesion motif in the hydrogel configured to reversibly adhere to and release the cells; and
a second immunomodulatory cargo encapsulated in the hydrogel.

2. The immunotherapy delivery system of claim 1, wherein the cell adhesion motif comprises a peptide configured to reversibly adhere to and release the cells.

3. The immunotherapy delivery system of any one of the claims above, wherein the cell adhesion motif is configured to bind to integrins on the cells.

4. The immunotherapy delivery system of any one of the claims above, wherein the cell adhesion motif comprises an arginine-glycine-aspartic acid (RGD) peptide.

5. The immunotherapy delivery system of any one of the claims above, wherein the nanoparticles comprise the cell adhesion motif.

6. The immunotherapy delivery system of any one of the claims above, wherein the nanoparticles are configured to present the cell adhesion motif.

7. The immunotherapy delivery system of any one of the claims above, wherein the cells comprise adoptive cells.

8. The immunotherapy delivery system of any one of the claims above, wherein the cells comprise chimeric antigen receptor (CAR) T cells or natural killer cells.

9. The immunotherapy delivery system of any one of the claims above, wherein the second immunomodulatory cargo comprises a protein.

10. The immunotherapy delivery system of any one of claims 1-8, wherein the second immunomodulatory cargo comprises a cytokine.

11. The immunotherapy delivery system of any one of claims 1-10, wherein the hydrogel comprises less than 5% polymer.

12. The immunotherapy delivery system of any one of claims 1-10, wherein the hydrogel comprises 1.5%-3% polymer.

13. The immunotherapy delivery system of any one of the claims above, wherein the hydrogel comprises approximately 2% polymer.

14. The immunotherapy delivery system of any one of the claims above, wherein the polymer comprises hydroxypropylmethylcellulose (HPMC).

15. The immunotherapy delivery system of any one of the claims above, wherein the polymer comprises hydroxypropylmethylcellulose (HPMC) with hydrophobic lipid dodecyl chains.

16. The immunotherapy delivery system of any one of the claims above, wherein the nanoparticles comprise poly(ethylene glycol)-bpoly(lactic acid) (PEG-PLA).

17. The immunotherapy delivery system of claim 16, wherein the nanoparticles comprise the cell adhesion motif attached to the poly(ethylene glycol)-bpoly(lactic acid) (PEG-PLA).

18. The immunotherapy delivery system of claim 17, wherein the nanoparticles comprise between a 10:90 and a 90:10 ratio of poly(ethylene glycol)-bpoly(lactic acid) (PEG-PLA) with a cell adhesion motif to poly(ethylene glycol)-bpoly(lactic acid) (PEG-PLA) without a cell adhesion motif.

19. The immunotherapy delivery system of claim 17, wherein the nanoparticles comprise between a 25:75 ratio and a 75:25 of poly(ethylene glycol)-bpoly(lactic acid) (PEG-PLA) with a cell adhesion motif to poly(ethylene glycol)-bpoly(lactic acid) (PEG-PLA) without with a cell adhesion motif.

20. The immunotherapy delivery system of any one of the claims above, wherein the hydrogel comprises 4-12% nanoparticles.

21. The immunotherapy delivery system of any one of the claims above, wherein the hydrogel is shear-thinning and self-healing.

22. The immunotherapy delivery system of any one of the claims above, further comprising a syringe or catheter containing the hydrogel.

23. A method of treating a disease, comprising:
delivering the immunotherapy delivery system of any one of the claims above to a patient; and
releasing the cells from the hydrogel into the patient.

24. The method of claim 23, further comprising releasing the cells from the hydrogel over a period lasting from one day to four weeks.

25. The method of claim 23 or 24, wherein the immunotherapy delivery system releases cells over the course of at least two weeks, at least three weeks, or at least four weeks.

26. The method of any one of claims 23-25, further comprising activating the cells with the second immunomodulatory cargo.

27. The method of any one of claims 23-26, further comprising expanding the number of cells in the hydrogel.

28. The method of any one of claims 23-27, wherein the disease is a solid tumor cancer.

29. The method of any one of claims 23-28, wherein the cells are autologous.

30. The method of any one of claims 23-28, wherein the cells are autogeneic.

31. The method of any one of claims 23-30, wherein the cells express a chimeric antigen receptor (CAR) that recognizes a tumor antigen.

32. The method of any one of claims 23-31, further comprising removing the cells from the patient or a donor;
isolating the removed cells;
expanding the number of cells in vitro;
modifying the removed and/or expanded cells; and/or
encapsulating the cells in the hydrogel, prior to the delivering step.

33. The method of any one of claims 23-32, wherein the cells successively attach to and detach from the cell adhesion motif in the hydrogel.

34. The method of any one of claims 23-33, wherein delivering comprises delivering the immunotherapy delivery system through a syringe or catheter.

35. The method of any of claims 23-34 wherein delivering comprises delivering the immunotherapy delivery system to the patient by a route selected from the group consisting of intravenous, intraperitoneal, intramuscular, intratumoral and subcutaneous.

36. The method of any of claims 23-35 wherein delivering the immunotherapy delivery system to the patient comprises delivering the system locally to a region of the patient needing treatment.

37. The method of any of claims 23-36 wherein delivering the immunotherapy delivery system to the patient comprises delivering the system to a solid tumor cancer in the patient.

38. The method of any of claims 23-35 wherein delivering the immunotherapy delivery system to the patient comprises delivering the system to a location remote from a region of the patient needing treatment.

39. The method of any of claims 23-35 or claim 37 wherein the disease comprises a solid tumor cancer and delivering the immunotherapy delivery system to the patient comprises delivering the system to a location in the patient remote from the solid tumor cancer.

40. The method of any of claims 23-35 or claims 37-39 wherein delivering the immunotherapy delivery system to the patient comprises delivering the system systemically to the patient.
